(19) United States
(12) Patent Application Publication
(10) Pub. No.: US 2017/0049822 A1
(43) Pub. Date: Feb. 23, 2017

(54) METHODS OF ENHANCING STEM CELL ENGRAFTMENT

(71) Applicant: TEXAS HEART INSTITUTE, Houston, TX (US)

(72) Inventors: Qi LIU, Houston, TX (US); Richard A.F. DIXON, Houston, TX (US)

(21) Appl. No.: 15/305,717

(22) PCT Filed: Apr. 22, 2015
(86) PCT No.: PCT/US2015/027099

§ 371 Date: Oct. 21, 2016

Related U.S. Application Data
(60) Provisional application No. 61/983,325, filed on Apr. 23, 2014.

(51) Int. Cl. A61K 35/28; A61K 31/5578

(52) U.S. Cl. CPC A61K 35/28; A61K 31/5578

(57) ABSTRACT

An effective amount of a composition comprising a stem cell, a stem cell engraftment enhancer, and a carrier fluid, for use in the treatment of an individual having a disease or at risk of developing a disease, wherein the disease is a vascular-associated disease and/or a muscular disease. A composition comprising PGI2-overexpressing human mesenchymal stem cells (PGI2-hMSCs), and a carrier fluid; wherein an effective amount of the composition is administered via a single treatment stream as an intramuscular injection to an individual having a disease or at risk of developing a disease, wherein the disease is a vascular-associated disease and/or a muscular disease, and wherein stem cell engraftment is enhanced in said individual by greater than about 200%, when compared to stem cell engraftment in an individual treated with a composition lacking the stem cell engraftment enhancer.

### CROSS-REFERENCE TO RELATED APPLICATION

The present application is the U.S. National Stage under 35 U.S.C. §371 of International Patent Application No. PCT/US2015/027099, filed Apr. 22, 2015, which claims priority to U.S. Provisional Patent Application No. 61/983,325, filed Apr. 23, 2014, the disclosures of which are hereby incorporated herein by reference.

### STATEMENT REGARDING SPONSORED RESEARCH

The invention described and claimed herein was made in part utilizing funds supplied by AHA NSDG, Grant #10SDG4260005.

### TECHNICAL FIELD

This disclosure relates to methods of enhancing stem cell engraftment. More specifically, it relates to compositions and methods of using biologically active compounds to enhance stem cell engraftment.

### BACKGROUND

The growing prevalence of peripheral arterial disease (PAD) is an increasing global concern as the population ages. PAD is an atherosclerotic disease associated with diabetes, hypertension, hypercholesterolemia, and coronary artery disease. Currently, PAD affects 12-14% of the general population, and its incidence is accelerating because of the increase in the elderly population. More than 10 million people in the United States have PAD. The two major clinical stages of PAD—intermittent claudication and critical limb ischemia (CLI)—result from insufficient blood supply to lower extremities, but the clinical outcome is more severe in the latter stage. Conventional treatments for PAD, such as angioplasty, stent deployment, and peripheral bypass surgery, are less effective when PAD progresses and causes obstruction of arterioles. In these cases, patients may develop untreatable claudication, rest pain, and ulcers that can progress to gangrene and other infections requiring amputation of a lower limb. Although surgical advancements have improved the lives of some PAD patients, many are not treated surgically because of the risk of complications. New therapeutic approaches are needed to promote vascular growth, reduce functional impairment of ischemic legs, and improve quality of life.

Exogenous prostacyclin (PGI2 or PGI₂) replacement therapy offers a therapeutic alternative for patients who are poor candidates for surgical revascularization, such as high-risk patients (e.g., the elderly). Clinical studies have shown that PGI2 therapy is efficacious, but because PGI2 is an unstable compound with a circulating half-life of 1-2 minutes, this approach requires continuous intravenous or intraarterial infusion, which is associated with side effects and several potential complications. While continuous intravenous PGI2 therapy is effective, this approach is inconvenient for PAD patients, as PGI2 must be administered by using a continuous pump with an indwelling catheter. This delivery system is cumbersome and greatly reduces the patient's quality of life. Moreover, significant adverse events are associated with this delivery system; infection at the infusion site can lead to life-threatening complications. In addition, continuous infusion of PGI2 is a financial burden. Although stable PGI2 analogues have been developed and used clinically, most still require continuous intravenous or subcutaneous infusion. An oral formulation of treprostinil was recently approved for pulmonary arterial hypertension (PAH) by the U.S. Food and Drug Administration (FDA), but its efficacy is minimal and must be used in combination with other agents and it has not been tested for PAD.

We have shown that a localized delivery approach in which a micro-osmotic pump is used to directly administer PGI2 analogue Carbaprostacyclin (CPGI2) to ischemic hindlimbs of mice may overcome the disadvantages of systemic PGI2 therapy. Local CPGI2 delivery alleviates hindlimb ischemia by improving perfusion and promoting arteriolar growth. However, there are side effects and potential complications associated with this therapeutic method as well.

A new approach to effectively deliver PGI2 is urgently needed for treating PAD patients. As such, there exists a need for improved compositions of local PGI2 delivery and methods of using same.

### BRIEF DESCRIPTION OF THE DRAWINGS

For a more complete understanding of the present disclosure and advantages thereof, reference will now be made to the accompanying drawings/figures in which:

FIG. 1 illustrates a schematic of biosynthesis of prostanoids (e.g., prostaglandins, such as prostaglandin D₂ (PGD₂), E₂ (PGE₂), F₂ (PGF₂), and I₂ (PGI₂) (prostacyclin), or thromboxane A₂ (TXA₂)) through coupling reactions of upstream cyclooxygenases (COXs) and downstream individual synthases;

FIG. 2A displays laser Doppler images of local treatment of mouse ischemic limbs with carbaprostacyclin (CPGI2) as compared to control (saline);

FIG. 2B displays a graph of a quantitative analysis of perfusion recovery of mouse ischemic limbs with CPGI2 treatment as compared to saline treatment;

FIG. 3 displays live images of distinct arterial growth of mouse ischemic limbs treated with CPGI2, wherein more intraarteriolar connections (solid line arrows) and corkscrew extensions of arterioles (dashed line arrows) developed in the CPGI2-treated versus the saline-treated group;

FIG. 4A displays a histogram of mean blood vessel size distribution in a quantitative micro-CT analysis;

FIG. 4B displays micro-CT images of microvascular network in CPGI2-treated and saline-treated ischemic legs; the red dashed circles show the vasculature of the thigh muscle where CPGI2 or saline was administered;

FIG. 5A displays western blot images of COX-1-10aa-PGIS and COX-1 expression in human mesenchymal stem cells (hMSC or hMSCs);

FIG. 5B displays a graph of PGI2 production levels in hMSCs engineered to overexpress PGI2 (PGI2-hMSCs) versus control;

FIG. 5C displays endothelial cell tube formation incubated with PGI2-hMSC conditioned medium;

FIG. 5D displays endothelial cell tube formation incubated with control medium;

FIG. 6A displays a schematic representation of the lentiviral vector encoding herpes virus thymidine kinase (HSV1-tk), mCherry fluorophore, and firefly luciferase reporter genes;

FIG. 6B displays representative in vitro bioluminescent imaging (BLI) images of hMSCs transduced with lentiviruses;

FIG. 6C displays a representative photomicrograph and its corresponding fluorescence image showing the expression of red mCherry fluorescent protein in transduced hMSCs;

FIG. 6D displays a graph of high efficiency lentiviral transduction in hMSCs as confirmed by flow cytometry analysis;

FIG. 7A displays representative BLI images of NOD-SCID mice 3 days after PGI2-hMSCs or 3.1-hMSCs were injected into the gastrocnemius muscle of the ischemic hindlimb;

FIG. 7B displays a quantitative analysis of the BLI images of FIG. 7A;

FIG. 8A displays BLI images of NOD-SCID mice over a 14 day period after PGI2-hMSCs or 3.1-hMSCs were injected into the gastrocnemius muscle of the ischemic hindlimb;

FIG. 8B displays a quantitative analysis of the BLI images of FIG. 8A;

FIG. 9A displays BLI images of NOD-SCID mice over a 5 day period after a hMSCs injection combined with daily cicaprost or CW501516 treatments;

FIG. 9B displays a quantitative analysis of the BLI images of FIG. 9A;

FIG. 10A displays a graph of systolic blood pressure in mice at 3 days after injection with PGI2-hMSC and 3.1-hMSC;

FIG. 10B displays a graph of mean arterial pressure in mice at 3 days after injection with PGI2-hMSC and 3.1-hMSC;

FIG. 11A displays a graph of functional recovery of ischemic hindlimbs in mice at 21 days after injection with PGI2-hMSC and 3.1-hMSC;

FIG. 11B displays a graph of functional recovery of ischemic hindlimbs in mice at 28 days after injection with PGI2-hMSC and 3.1-hMSC;

FIG. 12 displays endogenous Ki67⁺ cells spread within the hMSC injection area;

FIG. 13 displays confocal images indicating of endogenous proliferating (Ki67⁺) cells only rarely seen in regions further away from both 3.1-hMSC and PGI₂-hMSC injection site;

FIG. 14A displays representative confocal images of endogenous Ki67⁺Sca-1⁺ and Ki67⁺Sca-1⁻ cells;

FIG. 14B displays a quantitative analysis of Ki67⁺Sca-1⁺ cells surrounding PGI₂-hMSCs injection sites as compared to 3.1-MSC sites;

FIG. 14C displays a quantitative analysis of Ki67⁺Sca-1⁻ cells surrounding PGI₂-hMSCs injection sites as compared to 3.1-MSC sites;

FIGS. 15A-F display H19 RNA levels along with cell viability in C2C12 myoblasts in various coculture environments;

FIGS. 15G-I display H19 RNA levels along with cell viability in C2C12 myoblasts after specific knock down with H19 siRNA (H19 KD) compared to negative control siRNA;

FIGS. 16A-F display H19 RNA levels along with cell viability in primary myoblasts;

FIG. 16G-I display H19 RNA levels along with cell viability in primary myoblasts after specific knock down with H19 siRNA (H19 KD) compared to negative control siRNA; and

FIG. 16J displays representative images of H19 RNA fluorescence in situ hybridization in gastrocnemius muscle sections at 3 days after 3.1-hMSC or PG₂-hMSC injections.

### SUMMARY

Disclosed herein is an effective amount of a composition comprising a stem cell, a stem cell engraftment enhancer, and a carrier fluid, for use in the treatment of an individual having a disease or at risk of developing a disease, wherein the disease is a vascular-associated disease and/or a muscular disease.

Also disclosed herein is a composition comprising PGI2-overexpressing human mesenchymal stem cells (PGI2-hMSCs), and a carrier fluid; wherein an effective amount of the composition is administered via a single treatment stream as an intramuscular injection to an individual having a disease or at risk of developing a disease, wherein the disease is a vascular-associated disease and/or a muscular disease, and wherein stem cell engraftment is enhanced in said individual by greater than about 200%, when compared to stem cell engraftment in an individual treated with a composition lacking the stem cell engraftment enhancer.

Further disclosed herein is a composition comprising: human mesenchymal stem cells (hMSCs), Iloprost, and a carrier fluid; wherein the composition is administered to an individual having a disease or at risk of developing a disease, wherein the disease is a vascular-associated disease and/or a muscular disease, and wherein the composition is administered via multiple treatment streams comprising: a stem cell treatment stream, and a stem cell engraftment enhancer treatment stream; wherein the stem cell treatment stream comprises hMSCs and is administered via an intramuscular injection; and wherein the stem cell engraftment enhancer treatment stream comprises Iloprost and is administered via inhalation.

Further disclosed herein is a composition for stem cell engraftment, wherein the composition for stem cell engraftment comprises a stem cell, wherein the stem cell comprises human mesenchymal stem cells (hMSCs), endothelial progenitor cells (EPCs), hematopoietic stem cells (HSCs), cardiac progenitor cells (CPCs), satellite cells, or combinations thereof, a stem cell engraftment enhancer, wherein the stem cell engraftment enhancer comprises prostacyclin (PGI2), a PGI2 precursor, a peroxisome proliferator-activated receptor β/δ isoform (PPARδ) agonist, a cAMP inducer, a phosphodiesterase inhibitor, an endothelin receptor antagonist, a nitrous oxide modulating agent, a prostacyclin receptor (IP) agonist, a non-prostanoid IP receptor agonist, or combinations thereof, and a carrier fluid.

The foregoing has outlined rather broadly the features and technical advantages of the present invention in order that the detailed description of the invention that follows may be better understood. Additional features and advantages of the invention will be described hereinafter that form the subject of the claims of the invention. It should be appreciated by those skilled in the art that the conception and the specific embodiments disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

### DETAILED DESCRIPTION

It should be understood at the outset that although an illustrative implementation of one or more embodiments are provided below, the disclosed systems and/or methods may be implemented using any number of techniques, whether currently known or in existence. The disclosure should in no way be limited to the illustrative implementations, drawings, and techniques below, including the exemplary designs and implementations illustrated and described herein, but may be modified within the scope of the appended claims along with their full scope of equivalents.

Disclosed herein are embodiments of compositions for stem cell engraftment, designated a CSCE, and methods of using the same. For purposes of the disclosure herein, engraftment may be defined as (i) a process by which transplanted stem cells are retained within a tissue and/or (ii) a process by which upon transplantation of stem cells within a tissue, beneficial effects of stem cell transplantation (e.g., tissue healing; tissue repair; up-regulating lnc-RNA H19 in a host cell environment; host cell stimulation; improved exercise; etc.) are retained within the tissue, even when the stem cells themselves or a portion thereof are not retained within the tissue. In some embodiments, the CSCE may be used for targeted delivery of stem cells in specific body areas, wherein the stem cells may engraft and provide a repair function (e.g., a tissue repair function). In other embodiments, the CSCE may be used for targeted delivery of prostacyclin (PGI2 or PGI₂) in specific body areas, such as for example ischemic areas. While the current disclosure will be discussed in detail in the context of compositions for stem cell engraftment, it should be understood that other compositions for cell engraftment can comprise other types of cells, such as for example cells that have been engineered to produce prostacyclin (e.g., fibroblasts, endothelial cells, etc.). The cells can comprise any cells compatible with the disclosed methods and materials.

In an embodiment, the CSCE comprises a stem cell, a stem cell engraftment enhancer (designated a SEE), and a carrier fluid. In some embodiments, the stem cell may produce the SEE (e.g., PGI2). In other embodiments, the SEE may be supplied exogenously. Although the CSCEs will be discussed in detail in the context of peripheral arterial disease (PAD), it should be understood that treatment for other diseases is also contemplated, wherein enhanced engraftment of stem cells in the presence of a SEE may be useful.

As will be apparent to one of skill in the art, with the help of this disclosure, other suitable ingredients/components may be used in the CSCE, and each ingredient/component of the CSCE may perform more than one function (e.g., stem cells may be both the stem cell component as well as the SEE, wherein the stem cells may be engineered to express or overexpress the SEE). Each of the components of the CSCE as well as methods of using same will be described in more detail herein.

In an embodiment, stem cells may comprise stem cells and/or progenitor cells. In an embodiment, stem cells comprise natural stem cells, induced pluripotent stem cells, engineered adult stem cells, or combinations thereof. As will be appreciated by one of skill in the art, and with the help of this disclosure, natural stem cells refer to stem cells that are present in an organism (e.g., human) and may be isolated and used without further modification. Further, as will be appreciated by one of skill in the art, and with the help of this disclosure, induced pluripotent stem cells refer to stem cells (e.g., human adult stem cells) that have been modified (e.g., genetically modified) to provide pluripotent stem cells. Nonlimiting examples of stem cells suitable for use in the present disclosure include human mesenchymal stem cells (hMSC or hMSCs), endothelial progenitor cells (EPCs), hematopoietic stem cells (HSCs), cardiac progenitor cells (CPCs), satellite cells (e.g., myosatellite cells, skeletal muscle progenitor cells, etc.), or combinations thereof.

In an embodiment, the stem cells comprise hMSCs. Human mesenchymal stem cells offer advantages as vehicles for therapeutic gene transfer. Stem cell therapy is emerging as a novel and promising therapeutic approach for PAD. Clinical studies in PAD patients have shown that hMSCs are attractive candidates for stem cell-based strategies for tissue repair and gene therapy. hMSCs can be easily isolated and expanded to large numbers in vitro or ex vivo. Furthermore, hMSCs show low immunogenicity after allogeneic transplantation and provide paracrine factors for repairing damaged tissue. In addition, hMSCs accumulate at sites of injury to protect against inflammation and promote revascularization. These unique properties make hMSCs an excellent choice for exogenous gene delivery. hMSCs can be modified to express therapeutic genes before being administered directly to damaged tissues. This combined hMSC-gene therapy approach eliminates the need for repetitive or continuous gene delivery because hMSCs are able to self-renew.

In an embodiment, hMSCs may be engineered to augment production of specific desired proteins, thereby enhancing the therapeutic benefits provided by native hMSCs. In an embodiment, hMSCs may be engineered to produce PGI2, thereby offering a novel, targeted PGI2 replacement therapy for treating PAD, as will be described in more detail later herein.

EPCs generally comprise a population of rare cells that circulate in the blood or reside in vasculatures. EPCs have the ability to differentiate into endothelial cells (e.g., cells that make up the lining of blood vessels). In an embodiment, EPCs may be engineered to augment production of specific desired proteins, thereby enhancing the therapeutic benefits provided by native EPCs. In an embodiment, EPCs may be engineered to produce PGI2.

HSCs generally comprise a heterogeneous population of blood cells. HSCs are derived from mesoderm and have the ability to give rise to all the other blood cells. In an embodiment, HSCs may be engineered to augment production of specific desired proteins, thereby enhancing the therapeutic benefits provided by native HSCs. In an embodiment, HSCs may be engineered to produce PGI2.

CPCs generally comprise a population of resident cardiac stem cells. CPCs are thought to account for the physiological turnover of cardiac myocytes and vascular endothelial cells. In an embodiment, CPCs may be engineered to augment production of specific desired proteins, thereby enhancing the therapeutic benefits provided by native CPCs. In an embodiment, CPCs may be engineered to produce PGI2.

Satellite cells generally comprise small mononuclear progenitor cells with virtually no cytoplasm found in mature muscle. Satellite cells are precursors to skeletal muscle cells, able to give rise to satellite cells or differentiated skeletal muscle cells.

In an embodiment, the stem cells may be included within the CSCE in a suitable amount. In an embodiment the stem cells may be present within the CSCE in an amount of from about 5 million cells/mL to about 600 million cells/mL, alternatively from about 10 million cells/mL to about 500 million cells/mL, or alternatively from about 25 million cells/mL to about 400 million cells/mL, based on the total volume of the CSCE. In an embodiment the stem cells may be present within the CSCE in an amount of about 200 million cells/mL, based on the total volume of the CSCE. For purposes of the disclosure herein, the term “about,” when used in conjunction with a percentage or other numerical amount, means plus or minus 10% of that percentage or other numerical amount. For example, the term “about 400 million cells,” would encompass 400 million cells plus or minus 40 million cells.

In an embodiment, the CSCE can comprise a SEE. Generally, the SEE can enhance (e.g., increase) (a) an ability of the stem cells to engraft (e.g., be retained) in a tissue upon transplantation into the tissue and/or (b) retention of beneficial effects of stem cell transplantation (e.g., tissue healing; tissue repair; up-regulating lnc-RNA H19 in a host cell environment; host cell stimulation; improved exercise; etc.) in the tissue, even when the stem cells themselves or a portion thereof are not retained within the tissue. For purposes of the disclosure herein, a host cell refers to a cell present in a location (e.g., tissue location) where the stem cells are transplanted. Without wishing to be limited by theory, engraftment plays a role in co-stimulation of the host cells to proliferate and regenerate due to the stem cells being retained long enough to stimulate host cells and the new growth of muscle and blood vessels.

In an embodiment, the SEE may comprise PGI2; PGI2 stable precursors or analogues (e.g., Cicaprost, Iloprost, Beraprost, Carbaprostacyclin, Trepostinil, Epoprostenol, etc.); a peroxisome proliferator-activated receptor β/δ isoform (PPARδ) agonist (e.g., GW501516, also known as GW-501,516, GW1516, GSK-516, Endurobol, etc.); a cAMP inducer (e.g., forskolin, also known as coleonol, 8-bromo-cAMP, etc.); a phosphodiesterase inhibitor (e.g., sildenafil citrate (VIAGRA®), tadalafil (CIALIS®), vardenafil (LEVITRA®), etc.); an endothelin receptor antagonist (e.g., bosentan (TRACLEER®), ambrisentan (LETAIRIS®), macitentan (OPSUMIT®), etc.); a nitrous oxide modulating agent (e.g., nitrates, or soluble GMP cyclase inducers, such as for example riociguat (ADEMPAS)); a prostacyclin receptor (IP) agonist; a non-prostanoid IP receptor agonist (e.g., selexipag); and the like; or combinations thereof. While the current disclosure will be discussed in detail in the context of SEE comprising PGI2 and/or a PGI2 precursor, it should be understood that other classes of compounds (e.g., a PPARδ agonist, a cAMP inducer, a phosphodiesterase inhibitor, an endothelin receptor antagonist, a nitrous oxide modulating agent, an IP agonist, a non-prostanoid IP receptor agonist, etc.) may be used to enhance stem cell engraftment, thereby enhancing a repair function that such stem cells might exhibit.

In an embodiment, the SEE may be a biologically or pharmacologically active compound. For purposes of the disclosure herein, a biologically active compound can be defined as a compound that interacts in some fashion with any living cell, tissue, and/or organism. For example, PGI2, PGI2 precursors or analogues, PPARδ agonists, cAMP inducers, phosphodiesterase inhibitors, endothelin receptor antagonists, nitrous oxide modulating agents, IP agonists, and non-prostanoid IP receptor agonists are biologically active compounds.

In an embodiment, the SEE comprises PGI2. PGI2, a member of the prostaglandin family, is synthesized from arachidonic acid (AA) in a multistep process involving the enzymes cyclooxygenase-1 (COX-1) or cyclooxygenase-2 (COX-2) and prostacyclin synthase (PGIS). As a vasodilatory drug, PGI2 has multiple favorable properties for treating PAD. In addition to mediating vascular homeostasis, PGI2 inhibits thrombosis and platelet aggregation.

The function of PGI2 is primarily mediated by the PGI2 receptor (IP) on the cell surface. The role of PGI2 as an endogenous anti-thrombotic and vasodilative agent was confirmed with the experimental data generated in IP receptor-knockout mice. The IP-deficient mice developed without vascular problems in normal situations. However, an increased thrombotic tendency was observed in the IP-deficient mice when endothelial damage was induced. These findings indicate that the anti-thrombotic system mediated by PGI2 is activated in response to vascular injury to minimize the effects of vascular injury. It has been reported that defects in the IP receptor of platelets has pathogenetic significance for developing atherosclerosis at an early age. The evidence was derived from a 10 year-old human diagnosed with an occluded left popliteal artery who also had a defect of her IP receptor. This defect appears to be genetically determined and to contribute to the development of atherosclerosis.

In an embodiment, PGI2 may enhance functional benefits of human stem cell therapy. Accumulating evidence indicates a critical role for PGI2 in controlling stem cell recruitment and survival and in promoting angiogenesis. Patients with critical limb ischemia (CLI) have reduced numbers of circulating progenitor cells; however, after 4 weeks of treatment with a PGI2 analogue, such patients show increased levels of progenitor cells and pain relief. Human outgrown EPCs may produce PGI2 and endogenous secretion of PGI2 by EPCs may facilitate vascular regeneration. In contrast, inhibiting PGI2 production in EPCs may reduce their proliferation, survival, and angiogenic capacity in ischemic hindlimbs. PGI2 signaling promotes the migration and recruitment of EPCs to injured vessels. Impaired function of EPCs is associated with decreased endogenous PGI2 synthesis and signaling. PGI2 may have the ability to enhance the natural abilities of stem cells. The cell-protective property of PGI2 in vivo may attenuate cell loss by stimulating their plasticity to adapt to unfavorable environments.

In an embodiment, increasing or enhancing PGI2 biosynthesis in stem/progenitor cells may enhance the beneficial effects of stem cell therapy. Generally, biosynthesis, also known as biogenesis or anabolism, is a multi-step, enzyme-catalyzed process, wherein substrates are converted into more complex products. In biosynthesis, simple compounds are modified, converted into other compounds, or joined together to form macromolecules.

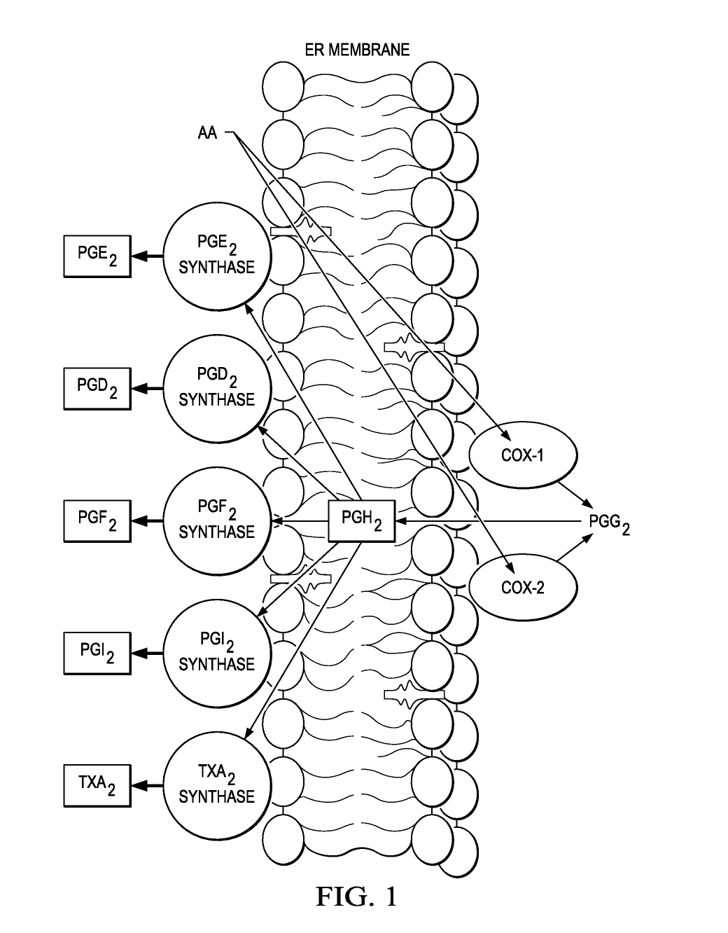

The recent discovery that COX-2 inhibitors may be linked to heart disease has greatly increased the interest in understanding the biology of COX enzymes, which convert a lipid molecule, AA, into different prostanoids (part of the eicosanoid family) having diverse and/or opposite biological functions. FIG. 1 shows a schematic of the biosynthesis of prostanoids. Biosynthesis of prostanoids generally comprises prostaglandins and thromboxane, formed via the COX pathway from arachidonic acid (AA) in three catalytic (tri-catalytic) steps (represented by some of the thin line arrows in FIG. 1). AA may traverse across an endoplasmic reticulum (ER) membrane (e.g., from a first or cytoplasmic side of the ER membrane to a second or luminal side of the ER membrane) and be converted in catalytic step 1 to prostaglandin G₂ (PGG₂) by COX isoform-1 (COX-1) and/or COX-2, wherein COX-1 and COX-2 may be located on the luminal side of the ER membrane. In catalytic step 2, PGG₂ may be further converted to prostaglandin endoperoxide (prostaglandin H₂ (PGH₂)) by COX-1 and/or COX-2. PGH₂ may traverse across the ER membrane (e.g., from the luminal side of the ER membrane to the cytoplasmic side of the ER membrane). In catalytic step 3, PGH₂ may be further isomerized to biologically active end-products (prostaglandin D₂ (PGD₂), E₂ (PGE₂), F₂ (PGF₂), and I₂ (PGI₂ (prostacyclin) or thromboxane A₂ (TXA₂) by individual synthases (PGD₂ synthase (PGDS), PGE₂ synthase (PGES), PGF₂ synthase (PGFS), and PGI₂ synthase (PGIS), or TXA₂ synthase (TXAS), respectively, as depicted in FIG. 1) in tissue specific manners, wherein such individual synthases may be located on the cytoplasmic side of the ER membrane. Prostanoids act as local hormones in the vicinity of their production site to regulate hemostasis and smooth muscle functions. Unlike the stable level of COX-1 expression, COX-2 expression is inducible and it responds to the stimuli of pro-inflammatory and other pathogenic factors. TXA₂ produced from PGH₂ by TXA₂ synthase (TXAS) has been implicated in various pathophysiological conditions as a proaggregatory and vasoconstricting mediator. PGI₂ is the main AA metabolite in vascular walls and has opposing biological properties to TXA₂, representing the most potent endogenous vascular protector acting as an inhibitor of platelet aggregation and a strong vasodilator on vascular beds. PGE₂ exhibits a variety of biological activities in inflammation. Aspirin and non-steroidal anti-inflammatory drugs (NSAID) inhibit both COX-1 and COX-2 activities to reduce the production of all prostanoids, which leads to thinning of the blood by reducing TXA₂ production and the suppression of inflammation through decreasing PGE₂ production. The selective COX-2 inhibiting drugs exhibit anti-inflammatory effects similar to aspirin and NSAIDs, but they may also promote strokes and heart attacks by decreasing the production of PGI₂, and increasing the production of TXA₂. This may occur because, when the COX-2 enzyme was specifically inactivated by COX-2 inhibitors, the PGH₂ produced by COX-1 was still available to be converted into other prostanoids such as TXA₂ by TXAS, leading to an increased risk of thrombosis and vasoconstriction.

Recently, PGI₂ has also been determined to be a ligand for the nuclear hormone receptor peroxisome proliferator-activated receptor (PPAR). Three PPAR-isoforms, PPARα, β/δ and γ have been cloned and implicated in the regulation of the expression of genes involved in lipid metabolism. In both skeletal and cardiac muscle cells it has been demonstrated that the metabolic conversion of fatty acids is under control by PPARs. PGI₂ and PGI₂ agonists (e.g., carbaprostacyclin, iloprost, etc.), can effectively induce DNA binding and transcriptional activation by PPAR. PGI₂, acting as a ligand for PPARδ, induces increased expression of PPARδ in the arterial wall after a balloon injury, suggesting that PGI₂ effects vasodilation and anti-platelet aggregation through the IP receptor and PPARδ. It has also been speculated that PGI₂, as a ligand for PPARδ, induces anti-inflammatory activity in vascular diseases, such as atherosclerosis.

In an embodiment, peroxisome proliferator-activated receptor-beta/delta (PPARδ) can be a potential regulator of PGI2 signaling. In the search for endogenous targets for PGI2 signaling, PPARδ was found to colocalize with COX-2/PGIS and actively respond to PGI2 agonists. PPARδ is a ligand-activated nuclear hormone receptor that is ubiquitously expressed in various tissues. It forms heterodimers with retinoid X receptor, which binds to the peroxisome proliferator response element in the promoter region of target genes to control transcription. Emerging evidence suggests that PPARδ plays a critical role in stem cell survival and neovascularization. Accordingly, activation of PPARδ by PGI2 may promote stem cell-mediated vascular regeneration in ischemic hindlimbs. Inhibition of PPARδ by selective antagonists or specific siRNA in human progenitor cells may reduce PGI2-induced regenerative ability and blood vessel formation. PGI2, in partnership with PPARδ, accelerates embryo implantation and blastocyst hatching. In addition to its pro-survival and pro-angiogenic roles, PPARδ is important in adaptive responses to environmental changes. As a metabolic sensor, PPARδ regulates several metabolic genes involved in cellular homeostasis. PPARδ may play a critical role in mitochondrial function. In an embodiment, PGI2-PPARδ axis may affect the ability of stem cells to adjust to environmental changes (e.g., may affect the viability of stem cells introduced to certain body areas, such as for example ischemic areas), thus might affect the ability of stem cells to engraft.

In an embodiment, the SEE comprises a PPARδ agonist, such as for example GW501516, also known as GW-501,516, GW1516, GSK-516, Endurobol, etc.

In an embodiment, the carrier fluids that may be used in the CSCE include any carrier fluid suitable for delivery of stem cells in vivo. In an embodiment, the carrier fluid comprises a pharmaceutically acceptable carrier. For purposes of the disclosure herein, a “pharmaceutically acceptable carrier” is meant to encompass any carrier that does not interfere with effectiveness of a biological activity of any active ingredient (e.g., stem cell, stem cell engraftment enhancer) and that is not toxic to an individual to which it is administered. “Pharmaceutically acceptable” as used herein adheres to the U.S. Food and Drug Administration guidelines.

In an embodiment, the CSCE may comprise an aqueous carrier fluid. In an embodiment, the aqueous carrier fluid comprises deionized water and a variety of additives that may promote the viability and health of the stem cells of the CSCE. In an embodiment, the carrier fluid comprises a saline solution (e.g., phosphate buffer saline).

Nonlimiting examples of additive suitable for use in the carrier fluid in the present disclosure include nutritional supplements, growth factors, proteins (e.g., human serum albumin or HSA), and the like, or combinations thereof. In an embodiment, the carrier fluid may be included within the CSCE in a suitable amount.

In an embodiment, PGI2 may be delivered by stem cells that may be engineered (e.g., programmed) to overexpress PGI2, e.g., express high levels of PGI2 or express PGI2 levels that are higher than the PGI2 levels expressed by the same stem cells prior to being engineered. A system that increases PGI2 biosynthesis in cells of the ischemic areas would help prevent the adverse events caused by conventional PGI2 delivery methods. As will be appreciated by one of skill in the art, and with help of this disclosure, effective and stable biosynthesis of PGI2 requires an increase in the expression of COX-1 or COX-2 in conjunction with PGIS, as illustrated in FIG. 1.

In an embodiment, the SEE may comprise a PGI2 precursor. In an embodiment, the PGI2 precursor may comprise a triple catalytic enzyme, a PGI2-overexpressing stem cell (PGI2-SC), a DNA sequence encoding for the triple catalytic enzyme, a cDNA sequence encoding for the triple catalytic enzyme, a host cell containing an expressible DNA sequence encoding for the triple catalytic enzyme, a vector comprising a DNA sequence encoding for the triple catalytic enzyme, a plasmid comprising a DNA sequence encoding for the triple catalytic enzyme, a fusion gene encoding for the triple catalytic enzyme, a synthetic PGI2 analogue, and the like, or combinations thereof.

Nonlimiting examples of synthetic PGI2 analogues suitable for use in the present disclosure include Iloprost, Carbaprostacyclin, Treprostinil, Cicaprost, Beraprost, Epoprostenol, and the like, or combinations thereof.

In an embodiment, stem cells such as hMSCs may be engineered to overexpress an active triple catalytic enzyme to promote PGI2 expression (e.g., release PGI2). In such embodiment, the PGI2 overexpression by hMSCs may provide a means for local PGI2 delivery in body areas such as ischemic areas (e.g., ischemic tissue) and may concurrently enhance the natural ability of hMSCs to mediate repair in ischemic tissue. Although local delivery of prostacyclin and/or prostacyclin analogues (e.g., carbaprostacyclin (CPGI2) may alleviate hindlimb ischemia by improving perfusion and promoting arteriolar growth, this approach is not clinically practical because an invasive catheter-connected pump carrying a prostacyclin and/or prostacyclin analogues solution is generally subcutaneously implanted. In an embodiment, a triple catalytic enzyme may enhance the expression of PGI2 in stem cells, such as for example hMSCs, EPCs, HSCs, CPCs, satellite cells, or combinations thereof.

Recent studies of the structure and function relationship of COX enzymes and PGIS have advanced knowledge of the molecular mechanisms involved in the biosynthesis of PGI2 in native cells. Crystallographic studies of detergent-solubilized COX-1 and COX-2 suggest that the catalytic domains of the proteins lie on the luminal side of the endoplasmic reticulum (ER) and are anchored to the ER membrane by hydrophobic side chains of amphipathic helices A-D. These hydrophobic side chains of the putative membrane anchor domains also form an entrance to the substrate-binding channel and potentially form an initial docking site for the lipid substrate, AA. Recent progress in the topology and structural studies of human PGIS and TXAS have led to the proposal of models in which PGIS and TXAS have catalytic domains on the cytoplasmic side of the ER, opposite the orientation of COXs. In this configuration, the substrate channels of all three enzymes, COX, PGIS and TXAS, open at or near the ER membrane surface. The coordination between COXs and PGIS or TXAS in the biosynthesis of TXA₂ and PGI2 may be facilitated by the enzyme's anchoring in the lipid membrane. The physical distances between COXs and PGIS are very small. Consequently, it should be possible to create a single protein molecule containing COX and PGIS sequences with minimum alteration of both enzymes' folding and membrane topologies by extending the N-terminal membrane anchor domain of PGIS using a transmembrane sequence linked to the COX-1 or COX-2, which then adopts the functions of both enzymes of COX and PGIS. In this case, AA could be directly converted into the vascular protector, PGI2, with concurrently decreasing the production of the unwanted PGE₂ and TXA₂.

In an embodiment, the triple catalytic enzyme may be characterized by a formula COX-linker-ES, wherein COX comprises a cyclooxygenase (COX) amino acid sequence, such as for example COX-1 or COX-2; wherein ES comprises an eicosanoid-synthesizing (ES) enzyme amino acid sequence; wherein the linker comprises from about 10 to about 22 amino acid residues of a transmembrane sequence; wherein the linker may be disposed between the COX and the ES; and wherein the linker may directly connect the COX to the ES. In an embodiment, the triple catalytic enzyme comprises a hybrid protein or hybrid peptide.

In some embodiments, the linker (e.g., linker peptide) may function as a transmembrane linker in a cell, such that folding ability and function of each enzyme (e.g., COX, ES) of the triple catalytic enzyme may be substantially unaltered compared to the folding ability and function of respective native enzymes. As will be appreciated by one of skill in the art, and with the help of this disclosure, the linker is a peptide, since it comprises a relatively short sequence of amino acids. For purposes of the disclosure herein, the terms “linker” and “linker peptide” can be used interchangeably.

In an embodiment, the linker (e.g., linker sequence) comprises His-Ala-Ile-Met-Gly-Val-Ala-Phe-Thr-Trp (SEQ ID NO. 1) or His-Ala-Ile-Met-Gly-Val-Ala-Phe-Thr-Trp-Val-Met-Ala-Leu-Ala-Cys-Ala-Ala-Pro-Pro-Leu-Val (SEQ ID NO. 2). In certain embodiments, the linker sequence comprises residues 1-11, 1-12, 1-13, 1-14, 1-15, 1-16, 1-17, 1-18, 1-19, 1-20 or 1-21 of SEQ ID NO. 2. In some embodiments, the linker peptide provides approximately 10 Å separation between the catalytic sites of the COX and the ES enzyme. In an embodiment, the connected enzymes (e.g., COX, ES) are preferably capable of substantially normal folding and enzymatic activity compared to the native folding and enzymatic activity of the native COX and ES enzymes.

In an embodiment, the triple catalytic enzyme may be characterized by a faster turnover rate when compared to a mixture of the native COX and ES enzymes. The hybrid protein (e.g., COX-linker-ES) does not only possess the individual enzymes' activities, but has a faster turnover rate as compared to a mixture of separate COX and ES enzymes.

In an embodiment, the ES may comprise PGIS or a downstream synthase thereof. In an embodiment, the PGIS downstream synthase may comprise prostaglandin E synthase (PGES), prostaglandin D synthase (PGDS), or prostaglandin F synthase (PGFS). In an embodiment, the triple catalytic enzyme may be characterized by formulas COX-linker-PGIS, COX-linker-PGES, COX-linker-PGDS, or COX-linker-PGFS, wherein COX comprises COX-1 or COX-2. In such embodiment, the triple catalytic enzyme combines the enzymatic functions of COX (e.g., COX-1, COX-2) and ES (e.g., PGIS, PGES, PGDS, PGFS) in a single hybrid protein.

In an embodiment, the triple catalytic enzyme may be characterized by a formula COX-linker-PGIS, wherein COX comprises COX-1 or COX-2. In an embodiment, the COX-linker-PGIS may adopt the functions of COX and PGIS. In an embodiment, the COX-linker-PGIS may be able to continually convert AA into prostaglandin G₂ (catalytic step 1), prostaglandin H₂ (catalytic step 2) and prostacyclin (PGI2; catalytic step 3), wherein the catalytic steps have been described previously herein. Such conversion of AA into PGI2 may be even faster than coupling reactions using unlinked, co-expressed COX and PGIS.

In an embodiment, the triple catalytic enzyme may be characterized by a formula COX-1-linker-PGIS. In an embodiment, the triple catalytic enzyme may catalyze the three catalytic steps (e.g., three key reactions) in the biosynthesis of PGI2, thereby enhancing the expression of PGI2 (e.g., increasing the production of PGI2). In such embodiment, the triple catalytic enzyme links COX-1 to PGIS and catalyzes three key reactions for efficient production of PGI2 from AA.

In an embodiment, the COX-1-linker-PGIS protein may comprise an 130 kDa recombinant protein, wherein the recombinant protein may be constructed by linking together human cyclooxygenase (COX) isoform-1 (COX-1) and PGIS via a linker. In such embodiment, the linker may comprise from 10 to 22 amino acid residues of a transmembrane sequence, as previously described herein. In an embodiment, the COX-1-linker-PGIS protein may comprise COX-1-10aa-PGIS, wherein the linker comprises a 10 amino acid (10aa) transmembrane sequence (e.g., SEQ ID NO. 1). As will be appreciated by one of skill in the art, and with the help of this disclosure, some COX-2 inhibitors inhibit COX-2 activity but not COX-1 activity. Thus, the introduction of the COX-1-linker-PGIS hybrid protein to vascular systems is expected to help overcome the damage of the vascular functions caused by COX-2 inhibitors. In an embodiment, the triple catalytic enzyme may be characterized by a formula COX-1-10aa-PGIS.

In some embodiments, the triple catalytic enzyme may be chemically synthesized. In other embodiments, the triple catalytic enzyme may be recombinantly produced. The triple catalytic enzyme and methods of producing and/or using same are described in more detail in U.S. Publication No. 20100015120 A1, which is incorporated by reference herein in its entirety.

In an embodiment, PGI2 may be delivered by stem cells (SCs) that may be engineered (e.g., programmed) to express high levels of PGI2. Stem cells that overexpress PGI2 may be referred to as PGI2-SCs, such as for example PGI2-hMSCs, PGI2-EPCs, PGI2-HSCs, PGI2-CPCs, PGI2-(satellite cells), etc. In an embodiment, the PGI2 precursor comprises a PGI2-SC.

In an embodiment, the COX-linker-ES (e.g., COX-1-linker-PGIS) may be introduced in stem cells via any suitable transfection methods, such as nucleofection. Nucleofection is a nonviral transfection technique. As will be appreciated by one of skill in the art, and with the help of this disclosure, stable expression of COX-linker-ES may be confirmed via a variety of biochemical methods, such as for example Western Blot, genomic PCR, RT-PCR, and the like, or combinations thereof.

In an embodiment, SCs (e.g., PGI2-SCs) comprise a DNA sequence encoding for a COX, a transmembrane linker peptide, and an ES. In some embodiments, COX comprises COX-1. In other embodiments, COX comprises COX-2. In an embodiment, ES comprises PGIS. In an embodiment, the linker directly connects the COX to the ES. In an embodiment, SCs (e.g., PGI2-SCs) comprise a DNA sequence encoding for the triple catalytic enzyme, and such DNA sequence may be referred to as a “fusion gene.”

Generally, stem cells may be transfected by introducing a plasmid expressing the triple catalytic enzyme that links COX to ES (e.g., COX-1-10aa-PGIS). Such plasmid may comprise a promoter and an antibiotic resistance gene for selection of stable cell lines. Nonlimiting examples of promoters suitable for use in the present disclosure include a human cytomegalovirus promoter; endothelial-specific promoters (e.g., tie gene promoter, Tie2 gene promoter also known as Tek gene promoter, ICAM-2 (intercellular adhesion molecule-2) promoter, Flk-1 (fetal liver kinase-1) promoter, Flt-1 (fms-like tyrosine kinase) promoter, thrombomodulin promoter, vWf (von Willebrand factor) promoter, VE-cadherin promoter, etc.); cardiomyocyte specific promoters (e.g., alpha-MHC (myosin heavy chain) promoter; troponin promoter); smooth muscle cell specific promoters (e.g., SM22alpha promoter); human muscle specific promoter; human muscle creatinine kinase promoter; human α-skeletal actin promoter; human desmin promoter; human troponin I promoter; and the like; or combinations thereof. Nonlimiting examples of antibiotic resistance genes suitable for use in the present disclosure include a neomycin resistance gene (e.g., resistant to antibiotic G418); a puromycin resistance gene; an ampicillin resistance gene; a kanamycin resistance gene; a blasticidin resistance gene; a hygromycin resistance gene; a gentamicin resistance gene; a spectinomycin resistance gene; a streptomycin/spectinomycin resistance gene; and the like; or combinations thereof.

After transfection (e.g., nucleofection), transfected cells may be grown (e.g., cultured) for selection for a time period of from about 1 week to about 4 weeks. Then, cell clusters may be selected for further subculture, propagated and examined for PGI2 and/or COX-linker-ES expression. Subcultures that overexpress PGI2 comprise PGI2-SCs.

In some embodiments, a vector may comprise a DNA sequence encoding for the triple catalytic enzyme. In certain embodiments, the vector comprises an expression vector, such as for example a retrovirus, a lentivirus, an adenovirus, an adeno-associated virus, etc. In such embodiments, the DNA sequence encoding for the triple catalytic enzyme may be introduced into SCs (e.g., a host cell) via transduction.

In an embodiment, the SCs comprise a host cell containing an expressible DNA sequence encoding for the triple catalytic enzyme.

In some embodiments, the triple catalytic enzyme may be produced by a host cell containing an expressible DNA sequence encoding for the triple catalytic enzyme. In an embodiment, the host cell may be transfected with a vector comprising the DNA sequence encoding for the triple catalytic enzyme to produce host cell containing an expressible DNA sequence encoding for the triple catalytic enzyme. In some embodiments, the host cell comprises a SC. In other embodiments, the host cell does not comprise a SC. In such embodiments, the host cell may produce the triple catalytic enzyme. The host cell may be cultured under conditions suitable for expression of the DNA sequence encoding the triple catalytic enzyme, and then the triple catalytic enzyme may be recovered. In an embodiment, the triple catalytic enzyme comprises enzymatically active cyclooxygenase, transmembrane linker, and enzymatically active prostacyclin synthase.

In an embodiment, a cDNA may comprise a sequence encoding for the triple catalytic enzyme. In such embodiment, the cDNA may be used for COX gene therapy.

In an embodiment, the CSCE may be prepared via any suitable method or process. The components of the CSCE (e.g., stem cells, SEE, carrier fluid) may be combined using any mixing device compatible with the composition, e.g., that does not alter or destroy the CSCE components, such as the cells, etc. In an embodiment, the stem cells and/or SEE may be suspended in a saline solution comprising HSA. More details regarding stem cell preparation for administering as a treatment are available in Cytotherapy, 2010, 12(5), pp 684-691; and JAMA, 2011, 306(19), pp 2110-2119; each of which is incorporated by reference herein in its entirety.

In an embodiment, the CSCE may be used for the treatment of an individual having a disease or at risk of developing a disease, wherein the disease can be a vascular-associated disease and/or a muscular disease, and wherein the CSCE may be a pharmaceutical composition.

In an embodiment, the CSCE may be used for the treatment of an individual having a vascular-associated disease or at risk of developing a vascular-associated disease, wherein the CSCE may be a pharmaceutical composition. In an embodiment, the vascular-associated disease may comprise PAD, peripheral vascular disease, thrombosis, ischemia, CLI, heart attack, acute myocardial infarction, congestive heart failure, pulmonary arterial hypertension, acute lung injury, stroke, inflammation in an organ or vessel of a vascular system, chronic kidney disease, leukemia, bone marrow transplant, metabolic diseases, diabetes, and the like, or combinations thereof.

In an embodiment, the CSCE may be used for the treatment of an individual having a muscular disease or at risk of developing a muscular disease, wherein the CSCE may be a pharmaceutical composition.

In an embodiment, a method of treating an individual having a disease or at risk of developing a disease, wherein the disease can be a vascular-associated disease and/or a muscular disease, may comprise administering to the individual an effective amount of the CSCE, wherein the CSCE may be a pharmaceutical composition, to enhance stem cell engraftment in said individual, thereby ameliorating, deterring and/or preventing the disease in said individual. For purposes of the disclosure herein, an “effective amount” of CSCE may be defined as an amount of CSCE that produces a therapeutic response or desired effect (e.g., increase PGI2 levels in a body area) in some fraction of individuals to which it is administered.

In an embodiment, a method of treating an individual having a vascular-associated disease or at risk of developing a vascular-associated disease may comprise administering to the individual an effective amount of the CSCE, wherein the CSCE may be a pharmaceutical composition, to enhance stem cell engraftment in said individual, thereby ameliorating, deterring and/or preventing the vascular-associated disease in said individual.

In an embodiment, a method of treating an individual having a muscular disease or at risk of developing a muscular disease may comprise administering to the individual an effective amount of the CSCE, wherein the CSCE may be a pharmaceutical composition, to enhance stem cell engraftment in said individual, thereby ameliorating, deterring and/or preventing the muscular disease in said individual.

In an embodiment, a method of treating an individual having a disease or at risk of developing a disease, wherein the disease can be a vascular-associated disease and/or a muscular disease, may comprise administering to the individual a pharmaceutical composition comprising an effective amount of the CSCE, to enhance stem cell engraftment in said individual, thereby ameliorating, deterring and/or preventing the disease in said individual.

In an embodiment, a method of treating an individual having a vascular-associated disease or at risk of developing a vascular-associated disease may comprise administering to the individual a pharmaceutical composition comprising an effective amount of the CSCE, to enhance stem cell engraftment in said individual, thereby ameliorating, deterring and/or preventing the vascular-associated disease in said individual.

In an embodiment, a method of treating an individual having a muscular disease or at risk of developing a muscular disease may comprise administering to the individual a pharmaceutical composition comprising an effective amount of the CSCE, to enhance stem cell engraftment in said individual, thereby ameliorating, deterring and/or preventing the muscular disease in said individual.

In an embodiment, the CSCE may be a pharmaceutical composition. For purposes of the disclosure herein, a pharmaceutical composition generally refers to any composition that may be used on or in a body to prevent, deter, diagnose, alleviate, treat, and/or cure a disease in humans or animals.

In an embodiment, the stem cell engraftment in an individual treated with a CSCE may be enhanced by greater than about 200%, alternatively by greater than about 300%, alternatively by greater than about 400%, or alternatively by greater than about 500%, when compared to stem cell engraftment in an individual treated with an otherwise similar composition lacking the SEE. In an embodiment, the stem cell engraftment in an individual treated with a CSCE may be enhanced by from about 200% to about 500%, when compared to stem cell engraftment in an individual treated with an otherwise similar composition lacking the SEE. For purposes of the disclosure herein, stem cell engraftment may be defined as retention of the stem cells by a tissue, subsequent to administering stem cells to an individual.

In some embodiments, the components of the CSCE may be administered at the same time and via a single treatment stream (e.g., a single injection, such as intramuscular injection, intra-arterial injection, etc.). In other embodiments, the components of the CSCE may be administered at the same time and via multiple treatment streams. In yet other embodiments, the components of the CSCE may be administered at different times and via multiple treatment streams.

In an embodiment, a method of treating an individual having a disease or at risk of developing a disease, wherein the disease can be a vascular-associated disease and/or a muscular disease, may comprise administering to the individual an effective amount of the CSCE via a single treatment stream (e.g., single stream CSCE treatment). In some embodiments, the single stream CSCE treatment may comprise PGI2-SCs and a carrier fluid.

In an embodiment, a method of treating an individual having a vascular-associated disease or at risk of developing a vascular-associated disease may comprise administering to the individual an effective amount of the CSCE via a single treatment stream (e.g., single stream CSCE treatment). In some embodiments, the single stream CSCE treatment may comprise PGI2-SCs and a carrier fluid.

In an embodiment, a method of treating an individual having a muscular disease or at risk of developing a muscular disease may comprise administering to the individual an effective amount of the CSCE via a single treatment stream (e.g., single stream CSCE treatment). In some embodiments, the single stream CSCE treatment may comprise PGI2-SCs and a carrier fluid.

In other embodiments, the single stream CSCE treatment may comprise SCs, a SEE, and a carrier fluid; wherein the SEE may be a biologically active compound and wherein the SEE may be exogenously supplied to the SCs. In some embodiments, the single stream CSCE treatment may comprise SCs, a SEE comprising PGI2 and/or PGI2 precursor, and a carrier fluid; wherein the SCs do not overexpress PGI2; and wherein the PGI2 may be exogenously supplied to the SCs in the form of PGI2 and/or PGI2 precursor. In other embodiments, the single stream CSCE treatment may comprise SCs, a SEE comprising a PPARδ agonist, and a carrier fluid; wherein the PPARδ agonist may be exogenously supplied to the SCs.

In yet other embodiments, the single stream CSCE treatment may comprise engineered SCs, a SEE, and a carrier fluid, wherein the SEE may be a biologically active compound. In such embodiments, the single stream CSCE treatment may comprise PGI2-SCs, a SEE comprising PGI2 and/or PGI2 precursor, and a carrier fluid; wherein the SCs overexpress PGI2; and wherein PGI2 may also be exogenously supplied to the PGI2-SCs in the form of PGI2 and/or PGI2 precursor.

In an embodiment, a method of treating an individual having a disease or at risk of developing a disease, wherein the disease can be a vascular-associated disease and/or a muscular disease, may comprise administering to the individual an effective amount of the CSCE via multiple treatment streams. In such embodiment, the CSCE may be administered via a stem cell treatment stream and via a SEE treatment stream; wherein the stem cell treatment stream comprises stem cells and a carrier fluid; wherein the SEE treatment stream may comprise a SEE and a carrier fluid; and wherein the SEE may be a biologically active compound. For example, the CSCE may be administered via a stem cell treatment stream and via a PGI2 treatment stream; wherein the stem cell treatment stream comprises stem cells and a carrier fluid; and wherein the PGI2 treatment stream may comprise a PGI2 and/or PGI2 precursor and a carrier fluid. In an embodiment, the stem cell treatment stream may be administered prior to, concurrent with, and/or subsequent to administering the SEE treatment stream. In some embodiments, the SEE treatment stream may be administered concurrent with and subsequent to administering the stem cell treatment stream.

In an embodiment, a method of treating an individual having a vascular-associated disease or at risk of developing a vascular-associated disease may comprise administering to the individual an effective amount of the CSCE via multiple treatment streams. In such embodiment, the CSCE may be administered via a stem cell treatment stream and via a SEE treatment stream; wherein the stem cell treatment stream comprises stem cells and a carrier fluid; wherein the SEE treatment stream may comprise a SEE and a carrier fluid; and wherein the SEE may be a biologically active compound. For example, the CSCE may be administered via a stem cell treatment stream and via a PGI2 treatment stream; wherein the stem cell treatment stream comprises stem cells and a carrier fluid; and wherein the PGI2 treatment stream may comprise a PGI2 and/or PGI2 precursor and a carrier fluid. In an embodiment, the stem cell treatment stream may be administered prior to, concurrent with, and/or subsequent to administering the SEE treatment stream. In some embodiments, the SEE treatment stream may be administered concurrent with and subsequent to administering the stem cell treatment stream.

In an embodiment, a method of treating an individual having a muscular disease or at risk of developing a muscular disease may comprise administering to the individual an effective amount of the CSCE via multiple treatment streams, as disclosed herein.

In embodiments when PGI2-SCs may be administered to an individual, upon engraftment of such stem cells, the PGI2-SCs may consistently produce PGI2, which may then be secreted into surrounding areas/tissues.

In an embodiment, the PGI2-SCs may be locally injected (e.g., intramuscular injection) in ischemic areas or tissues, such as for example ischemic heart tissue, ischemic kidney tissue, ischemic limb tissue, ischemic lung tissue, ischemic brain tissue, ischemic pancreas tissue, and the like. In such embodiment, the PGI2-SCs may consistently release PGI2 and may engraft into the ischemic tissue, thereby enhancing tissue vascularization and restoring blood flow into at least a portion of the ischemic tissue. In an embodiment, the PGI2-SCs may comprise a vehicle for direct delivery of PGI2 to ischemic tissue.

In an embodiment, a method of treating an individual having a disease or at risk of developing a disease, wherein the disease can be a vascular-associated disease and/or a muscular disease, comprising administering to the individual an effective amount of a CSCE can further comprise up-regulating a long non-coding RNA H19 (lnc-RNA H19) in a host environment.

In an embodiment, a method of treating an individual having a muscular disease or at risk of developing a muscular disease comprising administering to the individual an effective amount of a CSCE can further comprise up-regulating a long non-coding RNA H19 (lnc-RNA H19) in a host environment.

In an embodiment, a method of treating an individual having a vascular-associated disease or at risk of developing a vascular-associated disease comprising administering to the individual an effective amount of a CSCE can further comprise up-regulating a long non-coding RNA H19 (lnc-RNA H19) in a host environment. In such embodiment, up-regulating the long non-coding RNA H19 in the host environment can promote host cell growth (e.g., can promote endogenous progenitor cell activity under a hostile environment, such as for example under tissue damage and/or ischemia). As will be appreciated by one of skill in the art, and with the help of this disclosure, a host environment refers to a cellular environment in a location (e.g., tissue location) where the stem cells are transplanted. Generally, long non-coding RNAs (lncRNAs) are an array of non-protein coding transcripts over 200 nucleotides long and have emerged as critical transcriptional or post-transcriptional regulators of cellular activity. The lncRNA H19 is a maternally imprinted gene that is abundantly expressed during embryonic development. After birth, H19 expression is reduced except in skeletal muscle. While up-regulation of H19 in myoblasts has been proposed to promote differentiation and myogenesis, the cytoprotective properties of H19 on progenitor cells have not been elucidated.

Without wishing to be limited by theory, H19 up-regulation can promote progenitor cell (e.g., myogenic progenitor cell) survival under hypoxia, as supported by targeted H19 knock down leading to an increase in nonviable cells. Further, without wishing to be limited by theory, H19 may act as an early regulatory element in augmenting cellular adjustment to environmental stress, thereby mobilizing protection mechanisms and increasing resistance to stress. Further, without wishing to be limited by theory, H19 may promote cellular proliferation by modulating downstream target genes.

In an embodiment, paracrine effects (e.g., paracrine signaling) of CSCE (e.g., PGI2-hMSCs) on progenitor cells can be achieved by modulating lncRNA H19. Generally, paracrine signaling is a form of cell-cell communication in which a cell produces a signal to induce changes in nearby cells, altering the behavior or differentiation of those cells.

In an embodiment, CSCE (e.g., PGI2-hMSCs) can induce up-regulation of H19 RNA levels in target cells (e.g., host environment, host cells, host cell environment etc.). In such embodiment, the up-regulation of H19 RNA in target cells can be accompanied by a simultaneous reduction in progenitor cell death.

In an embodiment, a method of treating an individual having a vascular-associated disease or at risk of developing a vascular-associated disease may comprise administering to the individual an effective amount of PGI2-hMSCs, to enhance stem cell engraftment in said individual, thereby ameliorating, deterring and/or preventing the vascular-associated disease. In such embodiment, the vascular-associated disease may comprise PAD and the PGI2-hMSCs may be administered by local injection (e.g., intramuscular injection) into the ischemic tissue.

In an embodiment, a method of treating an individual having a vascular-associated disease or at risk of developing a vascular-associated disease may comprise administering to the individual an effective amount of PGI2-hMSCs, to enhance stem cell engraftment in said individual, thereby ameliorating, deterring and/or preventing the vascular-associated disease. In such embodiment, the vascular-associated disease may comprise PAD and the PGI2-hMSCs may be administered by intra-arterial injection.

In another embodiment, a method of treating an individual having a vascular-associated disease or at risk of developing a vascular-associated disease may comprise administering to the individual an effective amount of hMSCs along with an effective amount of a PGI2 precursor, to enhance stem cell engraftment in said individual, thereby ameliorating, deterring and/or preventing the vascular-associated disease. In such embodiment, the vascular-associated disease may comprise PAD; the hMSCs may be administered by local injection (e.g., intramuscular injection) into the ischemic tissue; and the PGI2 precursor may comprise a PGI2 analogue, such as for example iloprost. In an embodiment, iloprost may be administered by inhalation.

In yet another embodiment, a method of treating an individual having a vascular-associated disease or at risk of developing a vascular-associated disease may comprise administering to the individual an effective amount of hMSCs along with an effective amount of a PGI2 precursor, to enhance stem cell engraftment in said individual, thereby ameliorating, deterring and/or preventing the vascular-associated disease. In such embodiment, the vascular-associated disease may comprise diabetes; the hMSCs may be administered by local injection (e.g., intramuscular injection) into ischemic limb tissue; and the PGI2 precursor may comprise a PGI2 analogue, such as for example iloprost. In an embodiment, iloprost may be administered by inhalation.

In an embodiment, the method of treating an individual having a disease or at risk of developing a disease, wherein the disease can be a vascular-associated disease and/or a muscular disease, as disclosed herein advantageously displays improvements in one or more outcomes when compared to a treatment method with an otherwise similar composition lacking the SEE. In an embodiment, stem cell engraftment in an individual treated with a CSCE may be increased when compared to stem cell engraftment in an individual treated with an otherwise similar composition lacking the SEE.

In an embodiment, stem cell engraftment in an individual treated with a CSCE may be increased when compared to stem cell engraftment in an individual treated with an otherwise similar composition lacking the PGI2 and/or the PGI2 precursor. In an embodiment, PGI2-SCs (e.g., PGI2-hMSCs) may advantageously display an enhanced ability to promote angiogenesis when compared to otherwise similar stem cells that lack the ability to overexpress PGI2.

In an embodiment, the method of treating an individual having a disease or at risk of developing a disease, wherein the disease can be a vascular-associated disease and/or a muscular disease, as disclosed herein may have several advantages over current standard PGI2 therapies. In an embodiment, when PGI2-SCs (e.g., PGI2-hMSCs) are used as a vehicle for PGI2 delivery, the treatment method may advantageously deliver PGI2 directly to ischemic tissue. In an embodiment, when PGI2-SCs (e.g., PGI2-hMSCs) are used as a vehicle for PGI2 delivery, the treatment method may advantageously and consistently provide a high level of PGI2 to ischemic tissues. In an embodiment, when PGI2-SCs (e.g., PGI2-hMSCs) are used as a vehicle for PGI2 delivery, the treatment method may advantageously enhance the capability of stem cells (e.g., hMSCs) to repair the damaged tissue.

In an embodiment, when PGI2-SCs (e.g., PGI2-hMSCs) are used as a vehicle for PGI2 delivery, the treatment method as disclosed herein may advantageously and effectively alleviate tissue ischemia and improve functional recovery. The PGI2-SCs (e.g., PGI2-hMSCs) as disclosed herein may advantageously provide a way to specifically increase the biosynthesis of the vascular protector PGI2 in ischemic tissue, and as such is believed to be an important development in pharmacology.

In an embodiment, PGI2-SCs (e.g., PGI2-hMSCs) may advantageously allow for direct in vivo synthesis of the potent vascular protector, PGI2, from AA with a high efficiency, which may be used to prevent and rescue patients from vascular-associated diseases (e.g., PAD, peripheral vascular disease, thrombosis, ischemia, CLI, heart attack, acute myocardial infarction, congestive heart failure, pulmonary arterial hypertension, acute lung injury, stroke, inflammation in an organ or vessel of a vascular system, chronic kidney disease, leukemia, bone marrow transplant, metabolic diseases, diabetes, etc.) and/or muscular diseases through specifically increasing PGI2 production in target areas, such as for example in ischemic tissue.

In an embodiment, the method of treating an individual having a disease or at risk of developing a disease, wherein the disease can be a vascular-associated disease and/or a muscular disease, with a CSCE (e.g., PGI2-SCs, PGI2-hMSCs, etc.) as disclosed herein may advantageously allow for improved recovery (e.g., tissue healing, tissue repair, etc.) of such individual when compared to recovery of an individual treated with an otherwise similar composition lacking the SEE, and for maintained the improved recovery after stopping therapy with the CSCE. As described herein, benefits achieved with PGI₂-hMSCs are superior to those seen with control hMSCs or iloprost, indicating that the combination of hMSCs and PGI₂ can advantageously result in greater tissue healing and repair. Without wishing to be limited by theory, functional recovery is not induced by prolonged presence of PGI₂-hMSCs but rather by an improved mobilization of the host response.

In an embodiment, the method of treating an individual having a disease or at risk of developing a disease, wherein the disease can be a vascular-associated disease and/or a muscular disease, with a CSCE (e.g., PGI2-SCs, PGI2-hMSCs, etc.) as disclosed herein may advantageously confer pro-survival benefits to host cells (e.g., proliferating myogenic progenitor cells). In such embodiment, the method can comprise up-regulating the lnc-RNA H19 in host cell environment (e.g., host cell stimulation).

In an embodiment, the method of treating an individual having a disease or at risk of developing a disease, wherein the disease can be a vascular-associated disease and/or a muscular disease, with a CSCE (e.g., PGI2-SCs, PGI2-hMSCs, etc.) as disclosed herein may advantageously retain benefits/effects of stem cell transplantation even when not all the transplanted stem cells are retained at a stem cell transplantation location. In such embodiment, the individual may advantageously display an improved ability to exercise even when not all the transplanted stem cells are retained at a stem cell transplantation location. Additional advantages of the CSCE and treatment methods of using same may be apparent to one of skill in the art viewing this disclosure.

### EXAMPLES

The embodiments having been generally described, the following examples are given as particular embodiments of the disclosure and to demonstrate the practice and advantages thereof. It is understood that the examples are given by way of illustration and are not intended to limit the specification or the claims in any manner. Unless otherwise specified, the following methodology was used for performing the experiments detailed in the following examples herein.

Transfection of Human Mesenchymal Stem Cells with a Plasmid Expressing a Novel Triple-Catalytic Hybrid Enzyme.

Human mesenchymal stem cells (hMSCs, between passages 3-4, Lonza, Switzerland) were transfected by electroporation (Human MSC Nucleofector Kit, Lonza) to introduce a plasmid pcDNA 3.1 (Invitrogen, Carlsbad, Calif.) or a pcDNA 3.1 expressing a triple-catalytic hybrid enzyme that links cyclooxygenase-1 (COX-1) to prostacyclin synthase (PGIS, the hybrid enzyme [COX-1-10aa-PGIS]). After nucleofection, cells were grown under G418 (200 μg/ml) selection, and confluent cell monolayers were harvested for evaluation of stable expression of the transgene COX-1-10aa-PGIS. hMSCs containing pcDNA 3.1 were referred to as 3.1-hMSCs and those containing pCOX-1-10aa-PGIS were referred to as PGI₂-hMSCs.

Genomic PCR, Western Blot, and Enzyme Immunoassays.

Genomic DNA was isolated and purified from native hMSCs, 3.1-hMSCs, and PGI₂-hMSCs according to the manufacturer's protocol (DNeasy Blood and Tissue Kit, QIAGEN, Germantown, Md.). PCR was performed using total DNA (200 ng/sample), COX-1-10aa-PGIS specific primers, and platinum Taq DNA polymerase (Invitrogen). Cell lysates prepared from hMSCs, 3.1-hMSCs, and PGI₂-hMSCs were used to assess the expression of fusion protein (COX-1-10aa-PGIS) by Western blot. In brief, 5 μg of protein was fractionated by SDS-PAGE (4%-20% gradient gel, Bio-Rad, Hercules, Calif.) and transferred onto a polyvinylidene fluoride (PVDF) membrane. The membrane was incubated with a primary antibody against COX-1 (Cayman Chemical, Ann Arbor, Mich.) followed by an HRP-conjugated anti-mouse secondary antibody (Sigma, St. Louis, Mo.). Protein signals were detected by using the ECL system (Thermo Scientific, Rockford, Ill.). To verify equal loading of each protein sample, we stripped the membranes and re-probed them with β-actin monoclonal antibody (Sigma).

The secretions of PGI₂ in the supernatants from native hMSCs, 3.1-hMSCs, and PGI₂-hMSCs were measured by using the 6-keto prostaglandin F1α enzyme immunoassay (6-keto prostaglandin F1α EIA kit, Cayman Chemical) according to the manufacturer's instructions. Briefly, the supernatant was collected after incubating cells (4×10⁴, N=5) with arachidonic acid (20 μm in MSC cell basal medium) for 20 minutes at 37° C. The absorbance was read using a microplate reader (Safire II, Tecan, Triangle Park, N.C.), and the concentration (pg/ml) of 6-keto prostaglandin F1α was calculated for each sample by using XFluor4 Safire II, V4.62n software.

Lentiviral Transduction of hMSCs.

Lentiviral transduction was performed on hMSCs and quantitative flow cytometry was used to assess the transduction efficiency. Because the lentiviral particles contain triple reporter genes, including herpes virus 1 thymidine kinase (HSV1-tk), mCherry fluorophore, and firefly luciferase, transduced cells were tracked by using multiple types of imaging modalities.

Mouse Unilateral Hindlimb Ischemia Model.

All animal procedures were conducted according to the University of Texas Health Science Center Animal Welfare Committee guidelines in accordance with the National Institutes of Health Guide for the Care and Use of Laboratory Animals. 20 NOD/SCID mice (NOD/ShiLtSz-Prkdc^{scid}/J, 11-12 weeks old; Jackson Laboratory, Bar Harbor, Me.) were randomly divided into 2 treatment groups (n=10 each): 3.1-hMSCs, or PGI₂-hMSCs. To create unilateral hindlimb ischemia, the left femoral artery was surgically ligated in mice anesthetized by isoflurane inhalation (2-4% isoflurane in oxygen). Specifically, 2 adjacent sutures were placed on the femoral artery, proximal to the origin of the femoral bifurcation, to interrupt flow. The incision was closed, and the mice were returned to their cages. At 24 hours after surgery, 4.5×10⁵ 3.1-hMSCs or PGI₂-hMSCs were injected into the gastrocnemius muscle of the ischemic hindlimbs.

Laser Doppler Perfusion Imaging.

Serial measurements of perfusion were performed with the use of a laser Doppler image device (Perimed AB, Germany) at 24 hours and 3, 5, 7, and 14 days after cell injections in the cell-treated groups and at the same time points in iloprost-treated mice. Perfusion was expressed as the perfusion ratio in the ischemic compared to the contralateral, non-manipulated leg.

Running Endurance.

At 21 and 28 days, all mice were challenged with acute exercise in a run-to-exhaustion study. Before running, mice were acclimated to the treadmill (Eco 3/6, Columbus Instruments, Columbus, Ohio, inclination+5°) for 1-2 hours and to the motor sound for 15 minutes. At the start, the belt was set at a slow speed (6 meters/min), and the treadmill velocity was increased 2 meters every 2 minutes for the initial 12 minutes and held constant (18 m/min) thereafter. Exhaustion was defined as the point when mice spent more than 10 consecutive seconds on the shock grid without trying to reengage the treadmill. Maximal running time and distances were recorded.

Immunofluorescence and Hematoxylin & Eosin Staining.

Mice were euthanized by CO₂ inhalation at 3 days (n=4 mice/group) after cell delivery, and the gastrocnemius muscle of the ischemic hindlimb was excised and processed. Cross sections of muscle tissue (6 μm) were incubated overnight at 4° C. with the following primary antibodies individually or in combination: anti-Ki67 (Abcam, Cambridge, Mass.) and anti-Sca-1 (Biolegend, San Diego, Calif.). The sections were then incubated with corresponding secondary antibodies: Alexa Fluor-647 donkey or anti-rabbit IgG, Alexa Fluor-488 goat anti-rabbit IgG, or Alexa Fluor-488 donkey anti-rat IgG (all from Invitrogen). Nuclei were counterstained with DAPI. A confocal laser scanning microscope (Leica TCS SP5II, Buffalo Grove, Ill.) was used to obtain fluorescence images of stained sections. Image processing and quantitative analysis were performed by using the ImageJ software (http://imagej.nih.gov/ij/). To quantify Ki67⁺Sca-1⁺ and Ki67⁺Sca-1⁻ cells, a total 10 high power fields were analyzed.

Bioluminescence Imaging.

Bioluminescence imaging was performed using the Xenogₑn IVIS 200 system (Xenogen, Alameda, Calif.). Mice were intraperitoneally injected with D-luciferin (150 mg/kg) and imaged for 15 seconds at 2 minute intervals until maximum photon levels were reached. The mice were scanned at 1 hour (n=2 mice/group) and 14 days (n=5 mice/group) after cell injections. Imaging signals were quantified in units of maximum photons/s/cm²/steridian (photons/s/cm²/sr). The minimal non-invasive visualized value was set at 1×10⁶ p/s/cm²/sr.

Human MSC and Myoblast Coculture.

Mouse primary myoblasts from NOD/SCID mice (11-12 weeks old) were isolated. Primary myoblasts (4×10⁴ cells) or mouse C2C12 myoblasts (5×10⁴ cells, ATCC, Manassas, Va.) were seeded in 24-well tissue culture plates and cultured with growth medium [GM; DMEM medium (ATCC) containing 10% FBS (ATCC) and 1% penicillin-streptomycin (Lonza)] in a 5% CO₂ incubator at 37° C. for 24 hours. Transwell inserts (0.4 μm pore size; BD Biosciences; San Jose, Calif.) containing either 3.1-hMSCs or PGI₂-hMSCs (5×10⁴ cells/well in C2C12 coculture or 4×10⁴ cells/well in primary myoblast coculture) were placed into each well. The cells were cocultured in GM in a hypoxic incubator (1.5% O₂, New Brunswick Galaxy 14 S, Eppendorf, Enfield, Conn.) for 24 or 48 hours. In parallel, C2C12 cells were grown in the absence of any treatment or in the presence of iloprost (100 nM). the inserts were removed and the myoblasts were processed for viability assays, RT-qPCR, or other analyses.

Cell Viability Assay.

Trypan blue exclusion assay was used to obtain counts of viable and nonviable myoblasts. The assay was performed according to the online protocol provided by Life Technologies (http://www.lifetechnologies.com/us/en/home/references/gibco-cell-culture-basics/cell-culture-protocols/trypan-blue-exclusion.html). All experiments were performed in quadruplicate in 3 independent experiments.

RT-qPCR. After the treatments described above, myoblasts were washed with cold DPBS and harvested for RNA isolation (RNase Plus Micro Kit, QIAGEN). Total RNA (2 μg) was reverse transcribed by using high capacity RNA-to-cDNA kit (Invitrogen) and T100 thermal cycler (Bio-rad, Hercules, Calif.). qPCR was performed by using TaqMan Gene Expression Master Mix (Invitrogen) and 7900HT Fast Real-Time PCR System (Life Technologies, Grand Island, N.Y.). H19 specific primers/probes and 18S rRNA endogenous control (VIC/MGB Probe) were purchased from Life Technologies. The relative expression of RNA was calculated using RQ Manager 1.2.1 (the ΔΔCt method). All experiments were performed in triplicate in 3 independent experiments.

H19 Knock Down.

RNAiMAX transfection reagent, silencer select H19 siRNAs (n253569, n253570), a negative control set, and opti-MEM medium were purchased from Invitrogen. H19 siRNA was transfected into myoblasts according to the manufacturer's procedures. Negative control siRNA was transfected in parallel. Mouse C2C12 myoblasts (2×10⁴/well) and primary myoblasts (4×10⁴ cells) were seeded and cultured with GM in a 5% CO₂ incubator at 37° C. for 24 hours (day 1) prior to transfection. H19 siRNA (n253569, 100 pmol/siRNA) was transfected into myoblasts at day 2 and then H19 siRNAs (n253569 and n253570, 100 pmol/per siRNA) at day 3 to ensure sufficient knock down. Six hours after the second transfection, the cells were transferred to a hypoxic incubator (1.5% 02) for an additional 42 hours before being harvested for analyses. All experiments were performed in triplicate in 3 independent experiments.

H19 RNA Fluorescence In Situ Hybridization (RNA-FISH).

Mice were euthanized by CO₂ inhalation at 3 days (n=4 mice/group) after cell delivery. Cryosections of the gastrocnemius muscle from cell-treated ischemic hindlimbs were used for RNA-FISH according to the protocol provided by Biosearch Technologies Inc (Petaluma, Calif.). An H19 probe labeled with fluorescein was designed against mouse H19 transcript by using the online Stellaris FISH Probe Designer version 4.0. The nucleotide sequences containing miR-675-3p and miR-675-5p were excluded from the design. An Olympus BX-51 microscope with an oil immersion lens (100×1.4 NA) and an Olympus DP70 digital camera were used to obtain the images. The light source was an X-cite 120PC Mercury lamp (EXFO).

Statistical Analysis.

The data were expressed as the mean±standard error of mean (SEM). To determine statistical significance among the three independent groups, a one-way analysis of variance was used. To determine statistical significance between two groups, a two-tailed t test (Graph Pad Prism 5) was used. P<0.05 was considered statistically significant.

### Example 1

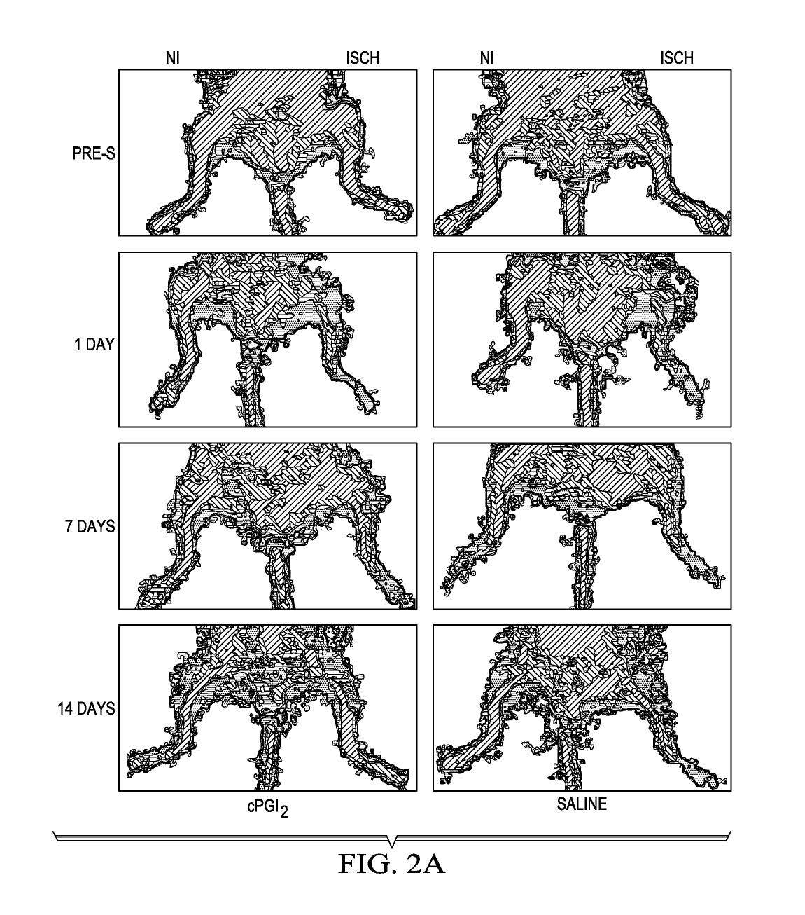

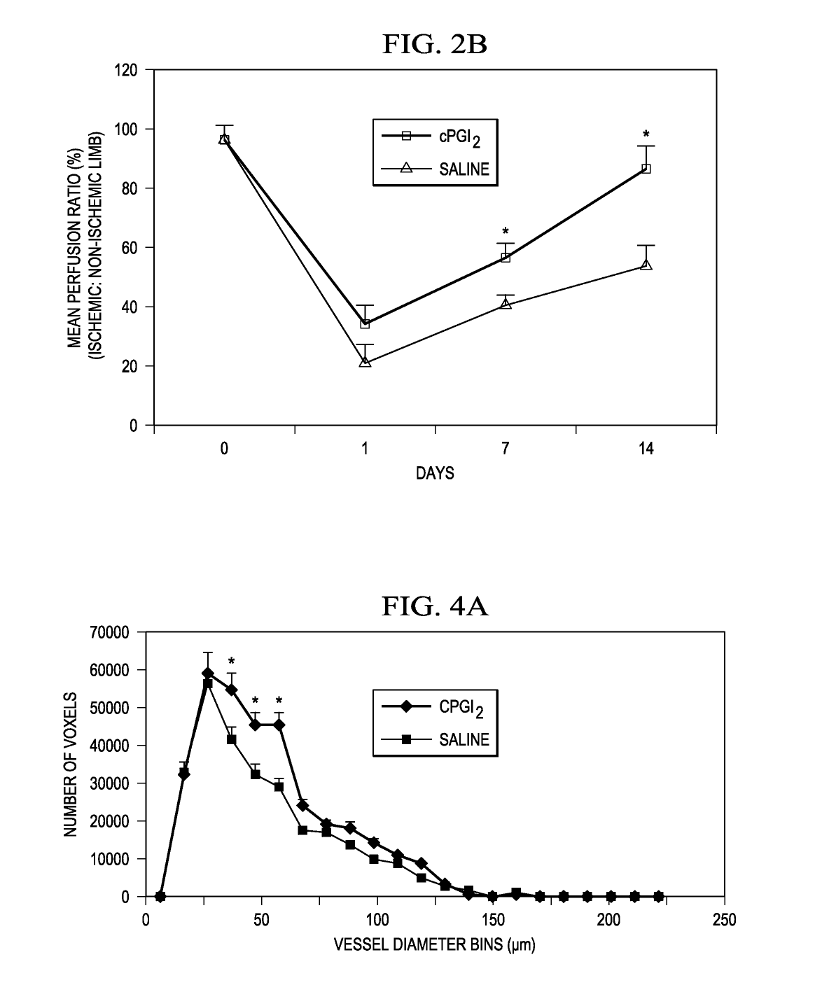

The outcomes of local delivery of CPGI2, a prostacyclin analogue, to ischemic hindlimbs were investigated. More specifically, blood perfusion in ischemic hindlimbs was monitored in conjunction with CPGI2 treatment. An osmotic pump was implanted and a catheter was used to deliver the prostacyclin analogue CPGI2 or saline (vehicle) to the surface of the ischemic anterior thigh muscle for up to 14 days. Blood perfusion was measured before ligation of femoral artery, 24 hours after drug treatment, and up to 14 days thereafter, and the results are shown in FIGS. 2A and 2B, wherein “NI” denotes “non-ischemic legs;” “Isch” denotes “ischemic legs;” and “Pre-S” denotes “before surgery.” FIG. 2A displays representative laser Doppler images that illustrate perfusion of ischemic (left) legs versus nonischemic contralateral limbs. FIG. 2B displays a quantitative analysis of ischemic hindlimb perfusion recovery following CPGI2 treatment. At 7 days, blood flow recovery was significantly better in mice treated with CPGI2 than in those treated with vehicle (58.80±5.74% versus 40.60±3.14%, respectively; *P<0.05; n=5/group; as shown in FIG. 2B). At 14 days, blood flow recovery in the CPGI2-treated group was significantly superior to that in the vehicle group (88.40±8.71% versus 54.60±6.67%, respectively; *P<0.01; n=5/group; as shown in FIG. 2B).

### Example 2

The outcomes of local delivery of CPGI2, a prostacyclin analogue, to ischemic hindlimbs were investigated. More specifically, arteriolar growth in ischemic hindlimbs was monitored in conjunction with CPGI2 treatment. Dynamic changes in the microvascular morphology of the limb region distal to the ligation where CPGI2 was applied were microscopically examined in live mice. At 7 days after treatment, structural remodeling at the arteriolar level in the CPGI2 group was more distinct than in the saline group, as shown in FIG. 3. FIG. 3 displays representative microscopic images that demonstrate increased vascular remodeling in ischemic legs treated with CPGI2 versus those treated with saline. More intra-arteriolar connections (solid line arrows, as shown in FIG. 3) and corkscrew extensions of arterioles (dashed line arrows, as shown in FIG. 3) developed in CPGI2-treated versus saline-treated groups. Arteriolar networks were identified by their branching out from a large feeder femoral artery or from a saphenous branch of descending genicular artery. Quantitative confocal analysis indicated that a number of microvessels ranging in size from 15-50 m in diameter was significantly higher in the CPGI2 group than in the vehicle group (38.00±2.41/high-power field [HPF] versus 18.69±2.12/HPF; P<0.01).

### Example 3

The outcomes of local delivery of CPGI2, a prostacyclin analogue, to ischemic hindlimbs were investigated. More specifically, remodeling of microvascular network in ischemic hindlimbs was monitored in conjunction with CPGI2 treatment. High-definition, volumetric, quantitative micro-CT was used to assess the overall microvascular geometry of ischemic and contralateral non-ischemic legs 14 days after femoral occlusion and constant local administration of CPGI2 or saline. Supporting the perfusion data in Example 1, it was found that a vascular volume of CPGI2-treated legs was significantly higher than that of saline-treated legs (41.28±2.22 versus 27.11±2.85 mm³; P<0.05). In addition, CPGI2-treated legs had significantly more blood vessels (0.16±0.014 versus 0.09±0.011 l/mm; P<0.05) and less distance between vessels (6.60±0.52 versus 10.15±1.14 mm; P<0.05) than did saline-treated legs. These findings suggest better development of the vascular system in the CPGI2-treated group than in the saline-treated group. Similar global morphometric analyses were used to evaluate contralateral non-ischemic legs. No significant differences were found between the CPGI2-treated and saline-treated groups in any of the 4 morphologic variables.

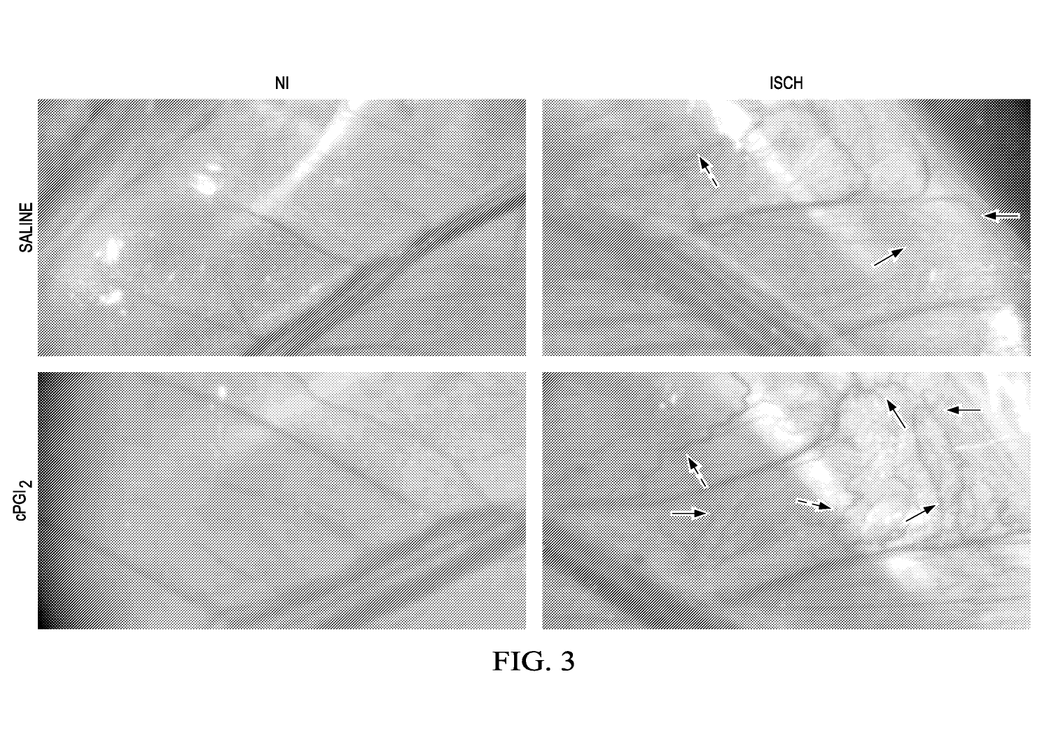

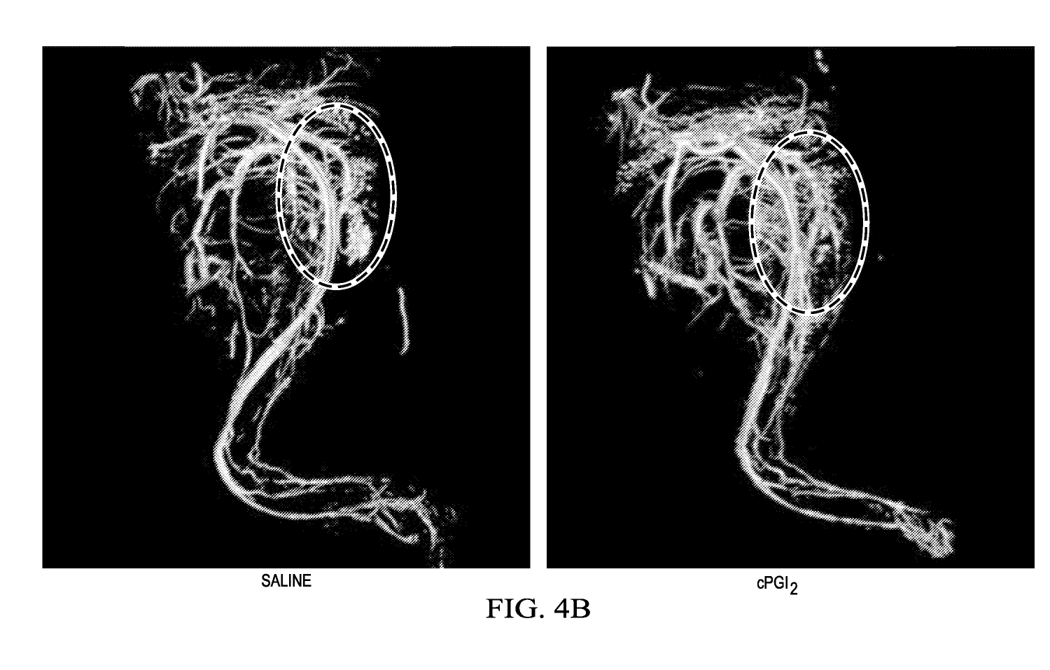

To further verify the pro-arteriogenic effect of CPGI2, a quantitative histogram was generated by using micro-CT to illustrate frequency and distribution of blood vessel size in CPGI2-treated legs in comparison to saline-treated or contralateral non-ischemic legs. Relative to saline-treated ischemic legs, CPGI2-treated ischemic legs showed a significant increase in small vessels, with vessel diameter bins ranging from 40-60 μm (P<0.05; as shown in FIG. 4A). FIG. 4A displays a histogram of mean blood vessel size distribution showing a marked increase in arterioles between 40-60 μm in the CPGI2-treated group. Representative micro-CT images of vessel remodeling in CPGI2-treated and saline-treated limbs showed that vascular remodeling is more prominent in the region of CPGI2 delivery than in the similar anatomic location of saline delivery, as seen in FIG. 4B. FIG. 4B displays representative micro-CT images of the microvascular network in CPGI2-treated and saline-treated ischemic legs. Referring to FIG. 4B, the red dashed circles show more vasculature in the thigh muscle where CPGI2 was administered as compared to the similar area where saline was administered. Contralateral non-ischemic legs were similarly evaluated and no significant differences in vessel distribution were found between the CPGI2-treated and saline-treated groups. Together, these data indicate that increased vessel formation is an important means by which CPGI2 improves perfusion in ischemic legs. CPGI2 treatment positively affects remodeling of the microvascular network in ischemic hindlimbs. Quantitative micro-CT analyses indicate CPGI2 augments vascular growth.

### Example 4

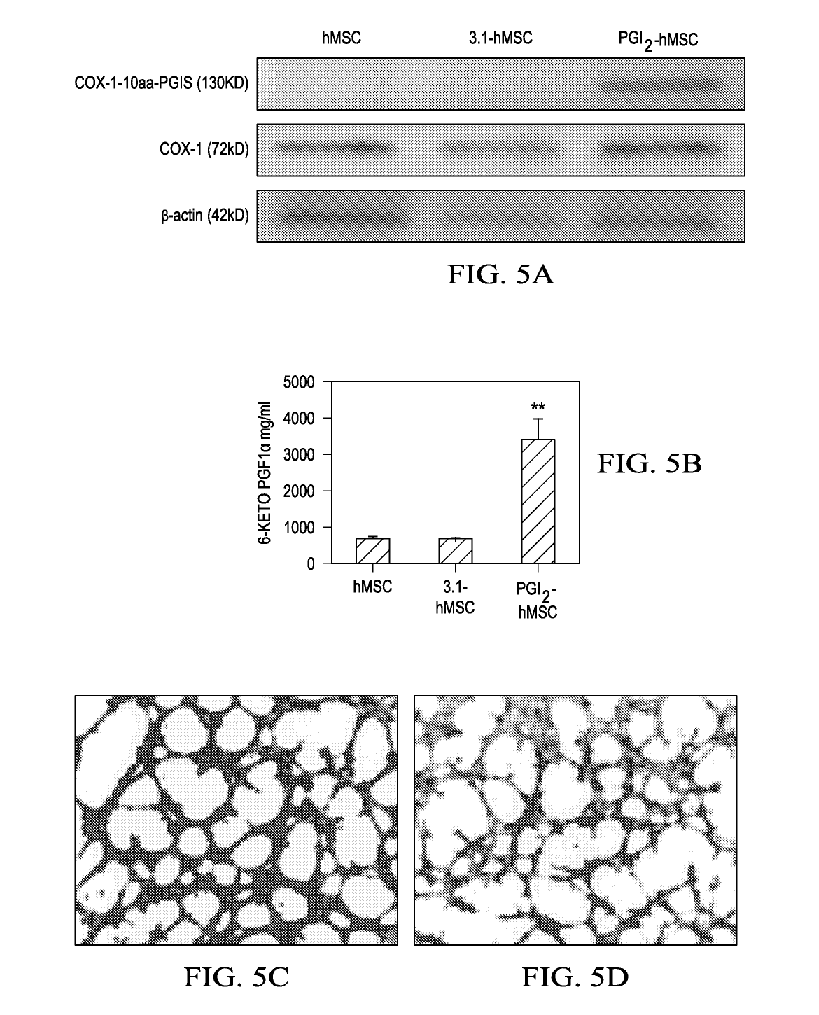

The ability of specifically engineered stem cells to overexpress PGI2 was investigated. More specifically, hMSCs were engineered to overexpress PGI2 and the ability of PGI2-hMSCs to promote angiogenesis was investigated. Although local delivery of CPGI2 alleviated hindlimb ischemia by improving perfusion and promoting arteriolar growth, as described in Examples 1, 2, and 3, this approach is not clinically practical because a catheter-connected pump carrying the CPGI2 solution was subcutaneously implanted on the mouse's back. A triple catalytic enzyme (e.g., COX-1-10aa-PGIS) that links COX-1 to PGIS and catalyzes 3 key reactions for the efficient production of PGI2 from arachidonic acid (AA, as illustrated in FIG. 1) was created, and the procedure is described in more detail in U.S. Publication No. 20100015120 A1. The effective and stable biosynthesis of PGI2 requires an increase in the expression of COX-1 or COX-2 in conjunction with PGIS. COX-1-10aa-PGIS was introduced into hMSCs via nucleofection, to produce PGI2-hMSCs. The stable expression of COX-1-10aa-PGIS in PGI2-hMSCs was confirmed by western blot, as shown in FIG. 5A. FIG. 5A displays western blots showing the overexpression of COX-1-10aa-PGIS fusion protein (130 kD) in PGI2-hMSCs and endogenous COX-1 protein levels in hMSCs.

To evaluate the production of PGI2 from engineered cells (e.g., PGI2-hMSCs), we used an enzyme immunoassay to measure the metabolite 6-keto PGF1α in the supernatant of cells that had been treated with arachidonic acid (20 μm) for 20 min. Compared with native hMSCs (containing no vector) and 3.1-hMSCs (containing pcDNA3.1, the vector used to construct pcDNA COX-1-10aa-PGIS), the concentration of 6-keto PGF1α was 5-fold higher in PGI2-hMSCs (**P<0.01, as seen in FIG. 5B). FIG. 5B displays a graph showing PGI2 production, which was notably higher in the supernatant of PGI2-hMSCs than in control cells.

To assess whether PGI2-hMSCs promote paracrine-related angiogenesis, endothelial progenitor cells we mixed either with conditioned medium (CM) from PGI2-hMSCs or 3.1-hMSCs. CM from PGI2-hMSCs markedly stimulated endothelial cell tube formation, as seen in FIG. 5C, as compared to endothelial cell tube formation in CM from 3.1-hMSCs as seen in FIG. 5D, indicating that the paracrine effects of PGI2 release included protective vascular activities. FIG. 5C displays a representative image of endothelial cell tubes incubated with CM from PGI2-hMSC, while FIG. 5D displays a representative image of endothelial cell tubes incubated with CM from 3.1-hMSC. Collectively, these findings confirmed successful establishment of hMSCs that consistently secrete high levels of PGI2 (PGI2-hMSCs).

### Example 5

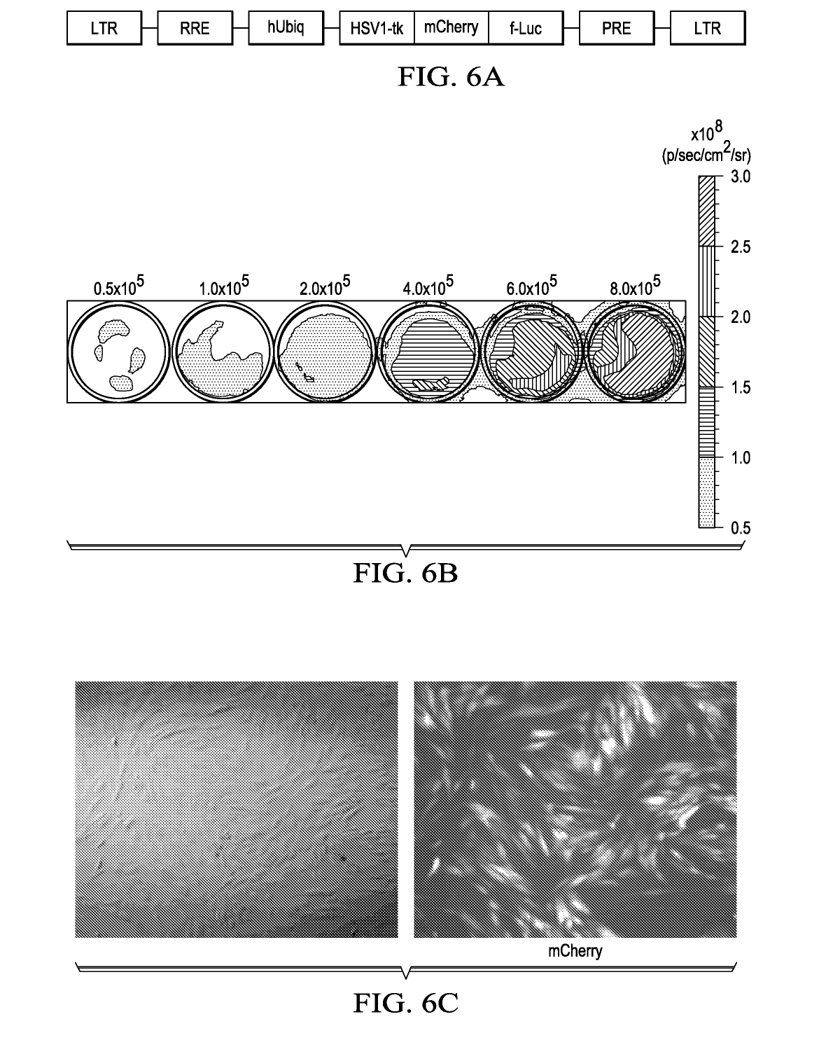

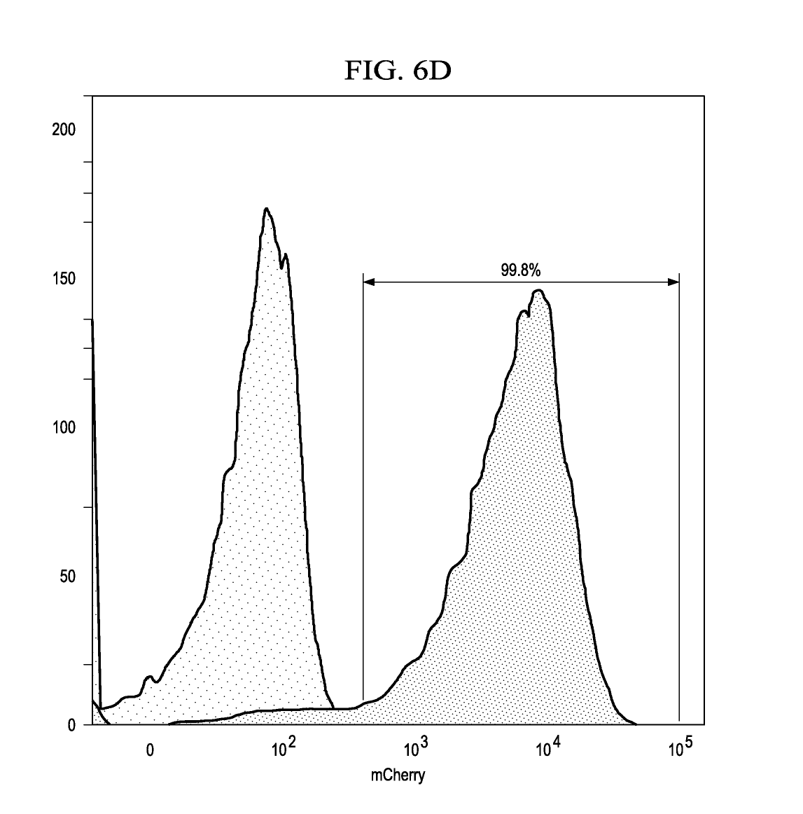

The ability of stem cells to engraft in ischemic tissues in the presence of PGI2 was investigated. More specifically, the ability of PGI2-hMSCs to engraft in ischemic hindlimbs was investigated. Hindlimb ischemia was created in NOD-SCID mice by performing unilateral surgical ligation using 2 adjacent sutures to interrupt the left femoral artery proximal to the origin of the femoral bifurcation. To evaluate the feasibility of administering cells locally, 4×10⁵ PGI2-hMSCs or equal numbers of vehicle (3.1-hMSCs) were injected directly into the gastrocnemius muscle of ischemic legs (n=5/group). The injected cells ubiquitously expressed human herpes simplex virus type 1-thymidine kinase, mCherry fluorophore protein, and firefly luciferase reporter genes driven by the human ubiquitin promoter, as shown in FIGS. 6A-6D. FIG. 6A displays a diagrammatic representation of the lentiviral vector encoding herpes virus thymidine kinase (HSV1-tk), mCherry fluorophore, and firefly luciferase genes; FIG. 6B displays representative in vitro bioluminescent imaging (BLI) images of hMSCs transduced with the lentiviral vector, wherein cells were consecutively diluted in a 6-well plate; it displays the positive relationship between bioluminescent intensity and cell numbers. FIG. 6C displays a representative photomicrograph and its corresponding fluorescence image showing the expression of red mCherry fluorescent protein in transduced hMSCs; and FIG. 6D displays a flow cytometry analysis graph confirming high efficiency of lentiviral transduction in hMSCs. hMSCs were efficiently transduced with a lentiviral vector containing triple fusion reporters (>99%). Direct correlation between the numbers of hMSCs and luciferase activity was confirmed, as shown in FIGS. 6A-6D. Luciferase catalyzes light-emitting photochemical reactions of luciferin in live cells, allowing for whole-body imaging to track the distribution and engraftment of transplanted cells.

In vivo BLI was performed using the Xenogen IVIS 200 system (Xenogen, Alameda, Calif.). For imaging, NOD/SCID mice were intraperitoneally injected with D-luciferin (150 mg/kg) and imaged for 15 sec, at 2 min intervals, until maximum photon levels were reached. The mice were scanned at 1, 3, 5, 7 and 14 day post-injection of 3.1-hMSCs or PGI2-hMSCs. Imaging signals were quantified in units of maximum photons/s/cm²/steridian (photons/s/cm²/sr).

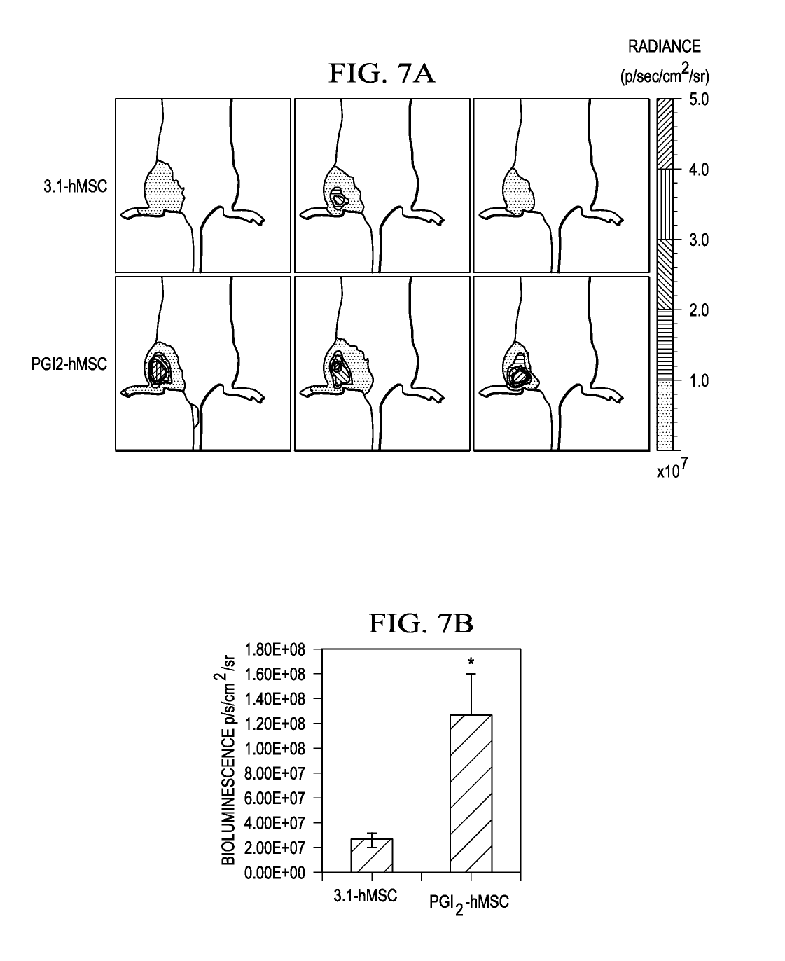

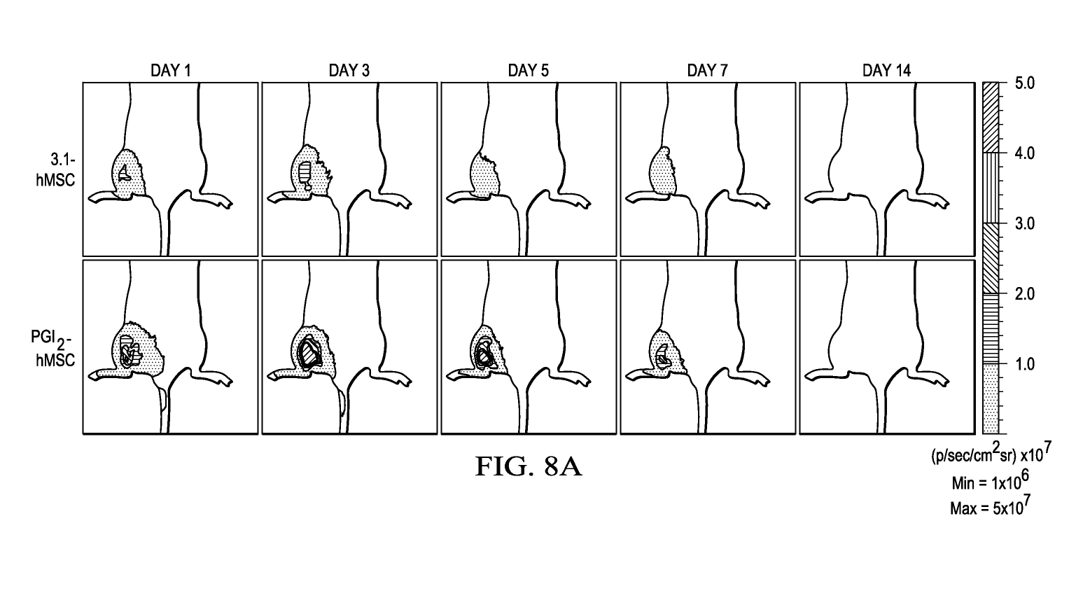

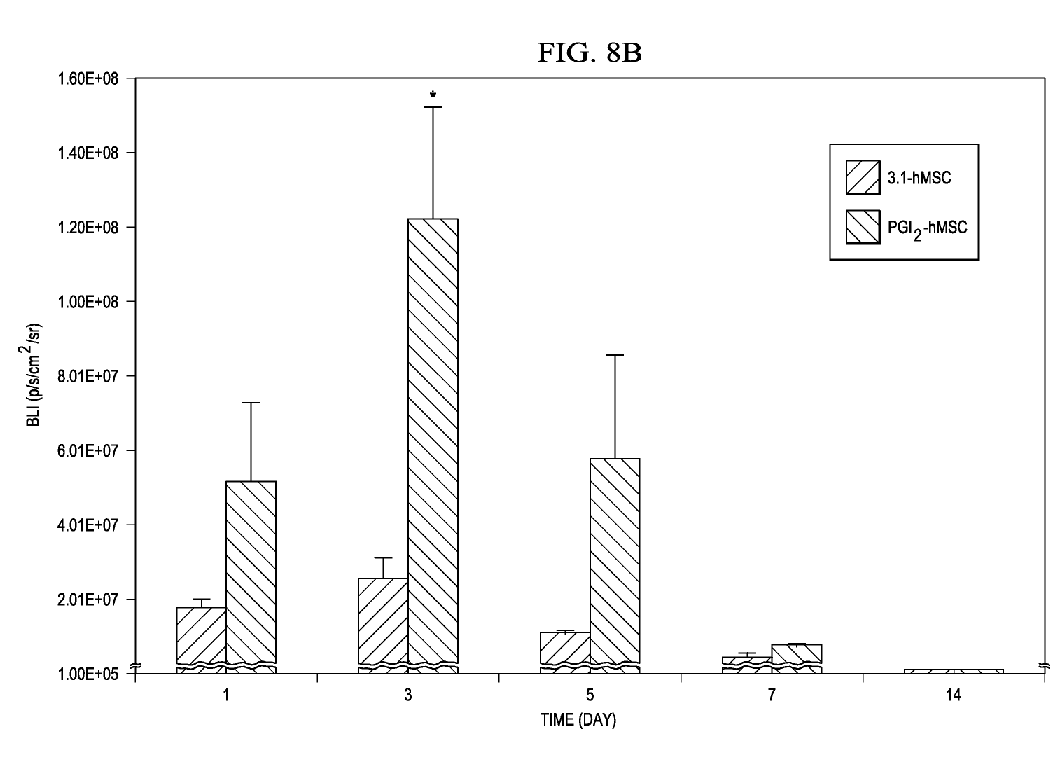

Signal measurement in whole-body images showed that bioluminescence was detected only in ischemic hindlimbs. Transplanted cells were not detected in other organs or tissues, as shown in FIGS. 7A and 8A. FIG. 7A displays representative in vivo BLI images of NOD-SCID mice 3 days after PGI2-hMSCs or 3.1-hMSCs were injected into the gastrocnemius muscle of the ischemic hindlimb. At 3 days after the cell injections, a markedly higher bioluminescent intensity was detected in ischemic hindlimbs that received PGI2-hMSCs than in those that received 3.1-hMSCs, as shown in FIG. 7B (12.20±3.05×10⁷ versus 2.49±0.58×10⁷ p/s/cm²/sr; PGI2-hMSCs versus 3.1-hMSCs, n=5/group, *P<0.05). FIG. 7B displays a quantitative analysis of the BLI images in FIG. 7A. These data showed a significant increase in the acute retention of PGI2-hMSCs in ischemic hindlimbs. Human mesenchymal stem cells engineered to secrete PGI2 show enhanced retention in ischemic hindlimbs.

FIG. 8A displays in vivo BLI images of NOD-SCID mice over a 14 day time period after PGI2-hMSCs or 3.1-hMSCs were injected into the gastrocnemius muscle of the ischemic hindlimb. The BLI signal was the strongest at 3 days after the cell injections, and it started to decay after day 3. At all time points over the first 7 days, a markedly higher bioluminescent intensity was detected in ischemic hindlimbs that received PGI2-hMSCs than in those that received 3.1-hMSCs, as shown in FIG. 8B (n=5/group, *P<0.05). FIG. 8B displays a quantitative analysis of the BLI images in FIG. 8A. These data showed a significant increase in the acute retention of PGI2-hMSCs in ischemic hindlimbs.

### Example 6

The ability of stem cells to engraft in ischemic tissues in the presence of SEE was investigated. More specifically, the ability of hMSCs to engraft in ischemic hindlimbs in the presence of different SEEs was investigated. The studies were conducted in as described in Example 5, unless otherwise specified.

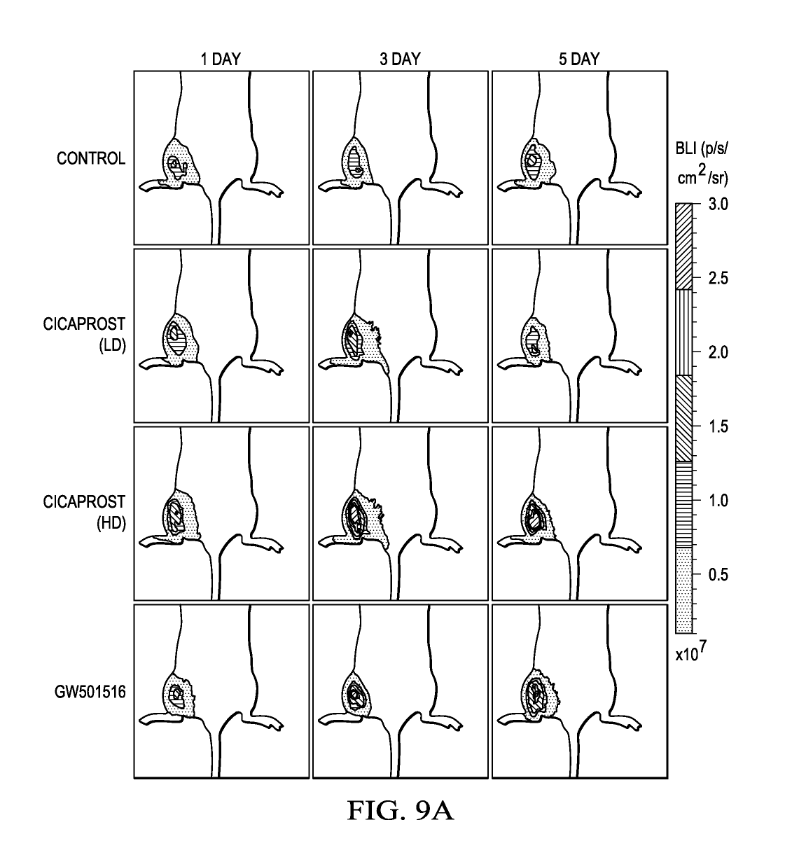

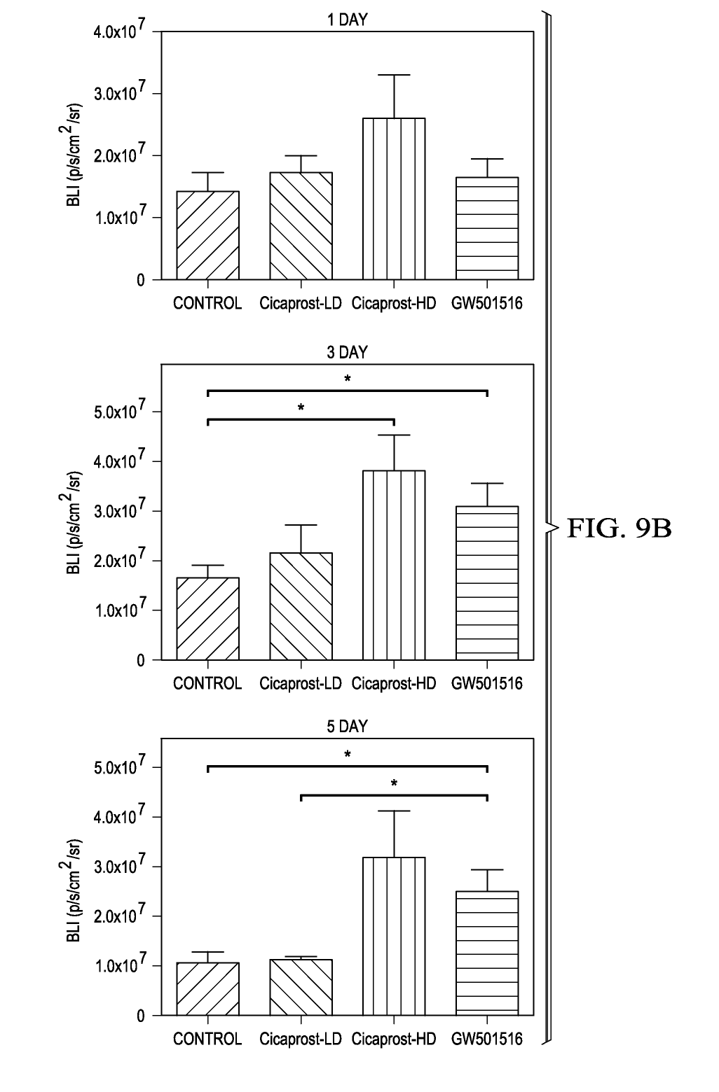

To investigate the signaling pathways involved in prostacyclin-induced cell retention after hindlimb ischemia, hMSCs were preconditioned with either prostacyclin receptor agonist Cicaprost (100 nM) or PPARβ/δ agonist GW501516 (100 nM) for 4 days in vitro. Cells were then injected into the gastrocnemius of ischemic hindlimbs of NOD/SCID mice. After cell injection, mice were either treated daily with Cicaprost (oral gavage, LD=0.3 mg/kg/day; HD=1 mg/kg/day) or GW501516 (intra peritoneal (IP), 5 mg/kg/day). LD=low dose and HD=high dose. In vivo BLI was performed on cell-drug treated mice. The mice were scanned at 1, 3, and 5 day post-injection of cells and drug treatment, and the data is shown in FIG. 9A.

Signal measurement in whole-body images showed that bioluminescence was detected only in ischemic hindlimbs. Transplanted cells were not detected in other organs or tissues, as shown in FIG. 9A.

FIG. 9A displays in vivo BLI images of NOD-SCID mice at various time points (e.g., day 1, day 3, and day 5) after hMSCs were injected into the gastrocnemius muscle of the ischemic hindlimb and while SEE drug treatment was ongoing. At 3 days after the cell injections, a markedly higher bioluminescent intensity was detected in mice that were treated with either Cicaprost or GW501516, when compared to the mice that received no drug treatment (e.g., control mice), as shown in FIG. 9B (n=5/group, *P<0.05). FIG. 9B displays a quantitative analysis of the BLI images in FIG. 9A. These data showed a significant increase in the acute retention of hMSCs in ischemic hindlimbs in the presence of either Cicaprost or GW501516, when compared to control mice that received no drug treatment. Exogenously supplied prostacyclin analogues improve hMSC retention in mouse hind limb ischemia model.

### Example 7

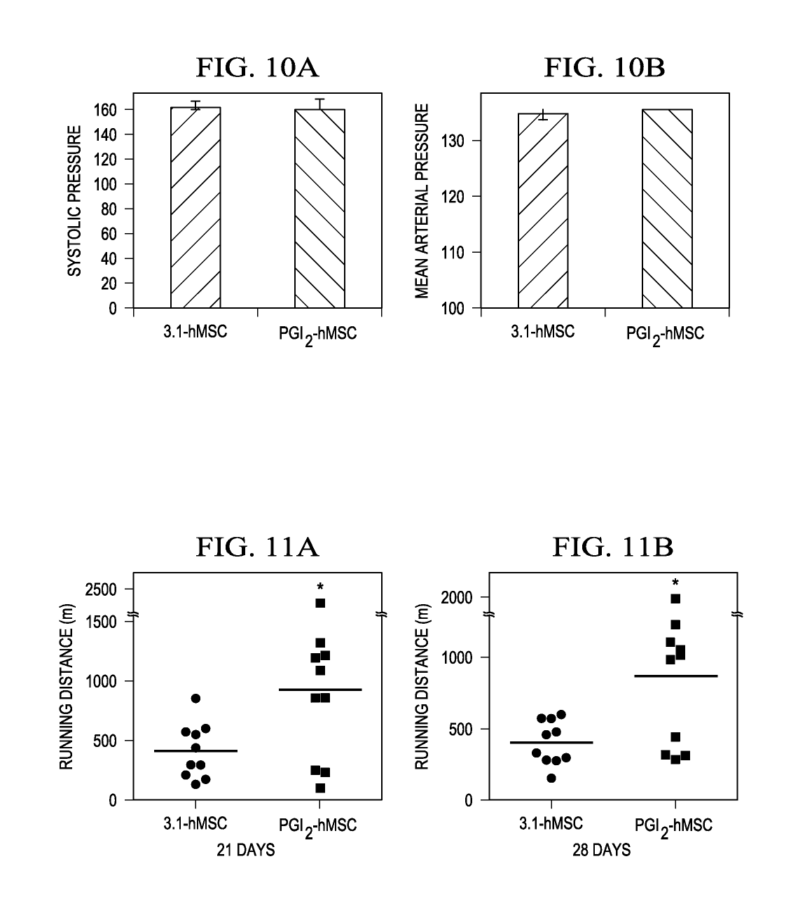

The systemic effects and safety of local PGI2-hMSCs treatment in mice was investigated. More specifically, the systolic blood pressure and mean arterial pressure in mice treated with PGI2-hMSCs was investigated. The major side effect of systemic infusion of PGI2 and its analogues is hypotension because PGI2 is a vasodilator. To ensure that local injection of PGI2-hMSCs does not induce hypotension, a non-invasive tail cuff method was used to measure systemic blood pressure 3 days after cell injection. Two physiologic parameters (systolic blood pressure and mean arterial pressure) were recorded by using a volume pressure recording sensor and a tail occlusion cuff (Coda 6; Kent Scientific Corp.). Hypotensive effects were not detected in PGI2-hMSC-treated mice, as shown in FIGS. 10A and 10B. FIG. 10A displays a graph of systolic blood pressure in PGI2-hMSC-treated and 3.1-hMSC-treated groups (N=5 mice/group), showing that the blood pressure was similar at 3 days after cell injection. FIG. 10A displays a graph of mean arterial pressure in PGI2-hMSC-treated and 3.1-hMSC-treated groups (N=5 mice/group), showing that the arterial pressure was similar at 3 days after cell injection. Local PGI2-hMSCs treatment did not significantly alter mouse blood pressure parameters.

### Example 8

Functional recovery of ischemic hind limbs after PGI2-hMSC was investigated. More specifically, the ability of PGI2-hMSC-treated and 3.1-hMSC-treated to run on a treadmill was investigated. Treadmill running was used to quantitatively measure the functional recovery of ischemic hindlimbs after cell therapy. PGI2-hMSC-treated and 3.1-hMSC-treated NOD/SCID mice were challenged with acute exercise (run-to-exhaustion). Before running, cell-treated mice were acclimated to the treadmill (Eco 3/6, Columbus Instruments, inclination+5°) for 1-2 hours and to the motor sound for 15 minutes. At trial start, the belt was set at a slow speed (6 meters/min), and the treadmill velocity was increased 2 meters every 2 minutes for the initial 12 minutes and held constant (18 meters/min) thereafter. Exhaustion was defined as mice spending more than 10 consecutive seconds on a shock grid without trying to reengage the treadmill. The maximal running time and distances were recorded, and the data is shown in FIGS. 11A and 11B. The mice were assessed at 21 and 28 days after cell delivery. FIG. 11A displays a graph of functional recovery of ischemic hindlimbs in mice at 21 days after injection with PGI2-hMSC and 3.1-hMSC, wherein the functional recovery is expressed as total distance run by a mouse (n=10/group, *P<0.05). FIG. 11B displays a graph of functional recovery of ischemic hindlimbs in mice at 28 days after injection with PGI2-hMSC and 3.1-hMSC, wherein the functional recovery is expressed as total distance run by a mouse (n=10/group, *P<0.05). The mice that were treated with PGI2-hMSC performed overall better at both 21 days and 28 days than the mice that were treated with 3.1-hMSC.

### Example 9

The ability of cells to accumulate at the site of PGI₂-hMSC injections was investigated. More specifically, the ability of endogenous Sca-1⁺Ki67⁺ and Sca-1⁻Ki67⁺ cells to accumulate at the site of PGI₂-hMSC injections in ischemic hindlimbs was investigated.

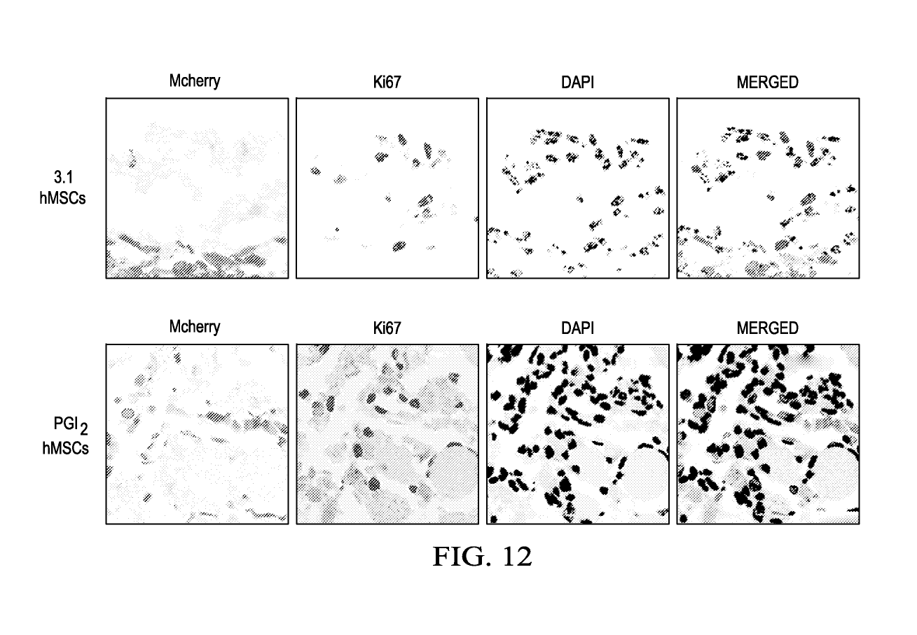

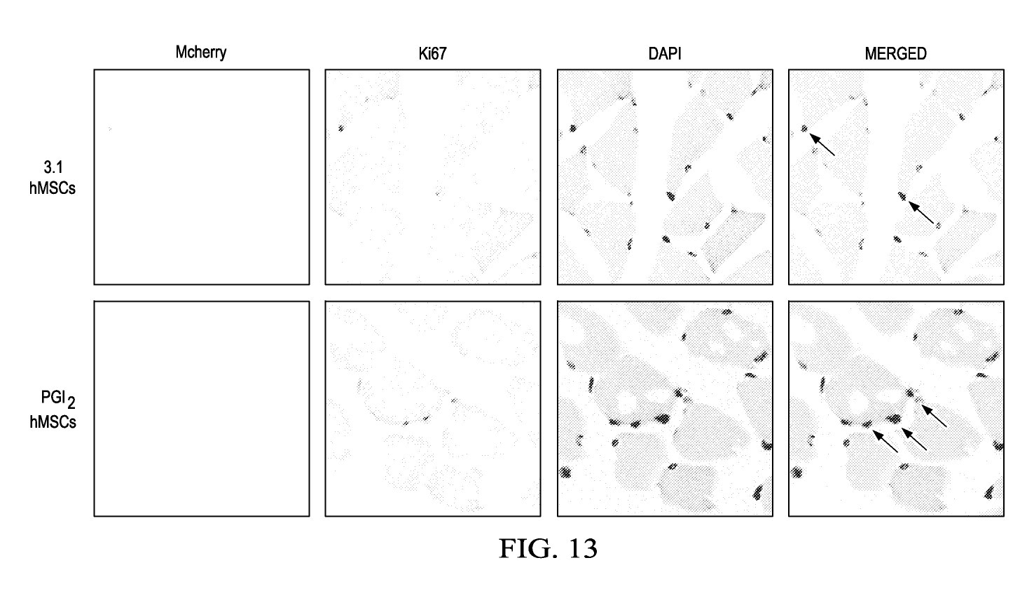

To determine if PGI₂-hMSCs induced an endogenous cellular response, we examined their effects in situ during hindlimb ischemia. Immunofluorescence staining of gastrocnemius muscle obtained 3 days after cell delivery showed the presence of cells positive for the proliferation-associated protein Ki67. Ki67⁺ cells tended to localize toward areas adjacent to the location of PGI₂-hMSCs (FIG. 12). FIG. 12 displays endogenous Ki67⁺ cells spread within the hMSC injection area at day 3. hMSCs contain red fluorescent mCherry protein. Ki67⁺ cells were observed only rarely in regions further away from PGI₂-hMSCs (>250 μm distance, FIG. 13). FIG. 13 displays confocal images indicating that endogenous proliferating (Ki67) cells were rarely detected in tissue that was located more than 250 μm away from the hMSC injection site at day 3 in both 3.1-hMSC and PGI₂-hMSC-treated mice.

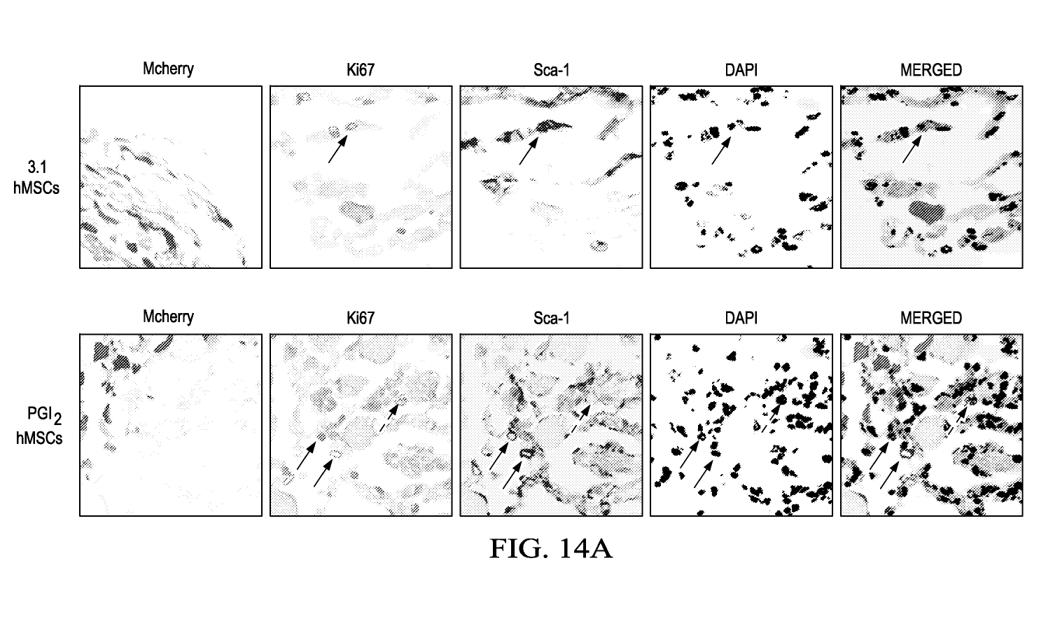

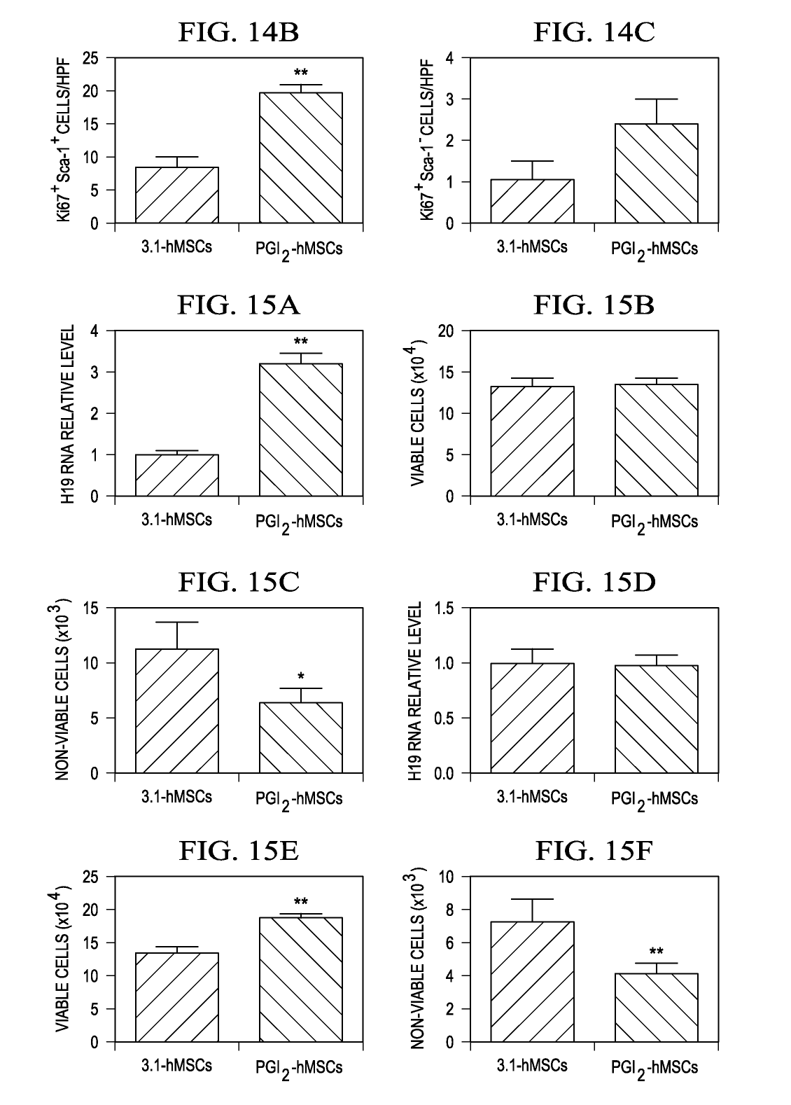

Notably, most Ki67⁺ cells expressed stem cell antigen-1 (Sca-1), a common marker on stem/progenitor cells. A similar anatomical distribution of endogenous Ki67⁺ and Sca-1⁺ cells was found in 3.1-hMSC-treated mice, although the number of cells positive for the markers was less than that seen with PGI₂-hMSC treatment (FIG. 14A). FIG. 14A displays representative confocal images illustrating the distribution of endogenous Ki67⁺Sca-1⁺ and Ki67⁺Sca-1⁻ cells.

When cell numbers were quantified in areas (125×125 μm²) surrounding injected hMSCs, the accumulation of Ki67⁺Sca-1⁻ cells was over 2-fold higher in PGI₂-hMSC-treated mice as compared to 3.1-hMSC-treated mice (P<0.01; FIG. 14B). FIG. 14B displays a quantitative analysis indicating significantly higher numbers of Ki67⁺Sca-1⁻ cells (**P<0.01) surrounding PGI₂-hMSCs injection sites as compared to 3.1-MSC sites. Similarly, in the same areas, the number of Ki67⁺Sca-1⁻ cells was also 2-fold higher in PGI₂-hMSC treatment than in 3.1-hMSC treatment (P=0.083, FIG. 14C). FIG. 14C displays a quantitative analysis indicating no statistical difference between the two sites in the number of Ki67⁺Sca-1⁻ cells (P=0.083).

Thus, although PGI₂-hMSCs did not incorporate into host tissues to generate functional new cells long-term, their early retention within ischemic beds yielded higher numbers of proliferating endogenous progenitor cells than did treatment with control 3.1-hMSCs. Moreover, Ki67⁺ resident cells were located next to PGI₂-hMSCs, suggesting that PGI₂-hMSCs may affect host cell proliferation or survival through paracrine effects.

### Example 10

The ability of PGI₂-hMSCs to promote endogenous progenitor cell survival was investigated. More specifically, the ability of PGI₂-hMSCs promote survival by upregulating long noncoding RNA H19 in proliferating C2C12 myoblasts under hypoxia was investigated.

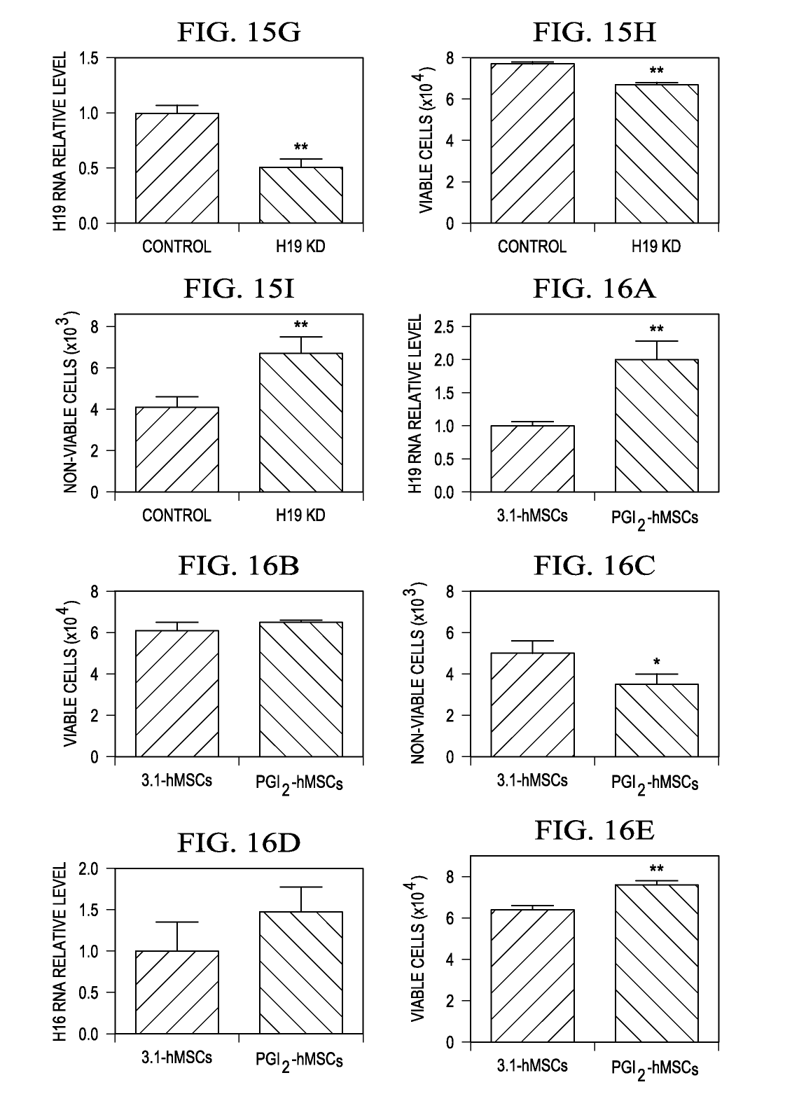

To gain insight into the paracrine effects of PGI₂-hMSCs, in vitro co-culture mechanistic studies were conducted under hypoxic conditions (1.5% O₂) that mimic the low-oxygen tension seen in ischemic hindlimbs. Myogenic progenitor cells (myoblasts) were used to assess the paracrine effects of PGI₂-hMSCs because greater exercise performance was observed at days 21 and 28 and muscle regeneration at day 14 in PGI₂-hMSC-treated mice. Emerging evidence has shown that long noncoding RNAs (lncRNAs) contribute significantly to cellular functions such as proliferation, survival, and differentiation. Specifically, the lncRNA H19 has been identified as an important factor in regulating muscle development. Thus, the effect of PGI₂-hMSCs on H19 transcript levels in myoblasts under hypoxic stress by coculturing proliferating C2C12 cells with PGI₂-hMSCs or 3.1-hMSCs (1.5% O₂) was assessed. At 24 hours, a 3-fold increase was found in H19 RNA in C2C12 cells cocultured with PGI₂-hMSCs as compared to those cocultured with 3.1-hMSCs (P<0.01, FIG. 15A). FIG. 15A indicates that H19 transcripts were significantly increased in C2C12 myoblasts after coculture with PG₂-hMSCs in a hypoxic incubator for 24 hours.

Coculturing with PGI₂-hMSCs had no obvious effects on C2C12 cell growth (13.61±0.41×10⁴ vs 13.45±0.50×10⁴ myoblasts cocultured with PGI₂-hMSC vs 3.1-hMSC, respectively, FIG. 15B) but caused a significant reduction of non-viable cells at this time point (trypan blue positive cells, 6.40±0.71×10³ vs 11.38±1.22×10³; P<0.05; FIG. 15C). FIG. 15B indicates that the number of viable C2C12 myoblasts was not different after 24 hours of coculture with PGI₂-hMSCs or 3.1-hMSCs, but PGI₂-hMSC coculture significantly reduced nonviable cells compared with 3.1-hMSC coculture at 24 hours (FIG. 15C).

At 48 hours, H19 RNA levels in C2C12 cells cocultured with PGI₂-hMSCs returned to levels similar to those cocultured with 3.1-hMSCs (FIG. 15D). FIG. 15D indicates that H19 RNA levels were comparable in C2C12 myoblasts after 48 hours of coculture with PG₂-hMSCs or 3.1-hMSCs in a hypoxic incubator.

However, PGI₂-hMSC coculture led to a marked growth of C2C12 myoblasts at 48 hours; significantly higher numbers of viable cells were detected in myoblasts cocultured with PGI₂-hMSCs than in those cocultured with 3.1-hMSCs (18.75±0.34×10⁴ vs 13.56±0.48×10⁴ cells; P<0.01; FIG. 15E). FIG. 15E indicates that coculture for 48 hours with PGI₂-hMSCs induced a significant increase of viable C2C12 cells. Moreover, nonviable myoblasts remained significantly lower in the PGI₂-hMSC cocultured group than in the 3.1-hMSC group (4.12±0.27×10³ vs 7.25±0.71×10³ cells; P<0.01; FIG. 15F). FIG. 15F indicates that coculture for 48 hours with PGI₂-hMSCs induced a significant decrease of nonviable cells.

Because of the reduction in C2C12 cell death and the concomitant increase of H19 RNA levels after 24 hours of coculture with PGI₂-hMSCs, the effects of H19 RNA on cell survival and proliferation were examined under hypoxia by knocking down H19 RNA in proliferating myoblasts. To achieve this, siRNA transfection was performed twice at 24-hour intervals and 2 silencer select small interfering RNAs (siRNAs) that target 2 regions of H19 during the second round of siRNA transfection were used simultaneously. A 50% reduction of H19 RNA levels as compared to negative control siRNA (P<0.01; FIG. 15G) was observed. FIG. 15G indicates that H19 RNA levels were significantly reduced in C2C12 myoblasts after specific knock down with H19 siRNA (H19 KD) compared to negative control siRNA.

H19 silencing caused a small, albeit significant, reduction in the number of viable cells at the end of 3 days of siRNA treatment (7.72±0.07 vs 6.67±0.10×10⁴ cells in negative control siRNA-transfected vs H19 siRNA-transfected myoblasts; P<0.01; FIG. 15H). FIG. 15H indicates that H19 silencing significantly reduced the numbers of viable cells. A concurrent increase of myoblast death was also detected after H19 siRNA treatment (4.12±0.27 vs 6.70±0.45×10³ cells, negative control siRNA vs H19 siRNA-transfected cells; P<0.01; FIG. 15I). FIG. 15I indicates that H19 silencing increased cell death. Together, in vitro data clearly suggest that the secretome of PGI₂-hMSCs provides cytoprotection by upregulating H19 RNA levels in progenitor cells and that silencing H19 affects cell survival/growth.

### Example 11

The ability of PGI₂-hMSCs to induce H19 lncRNA upregulation was investigated. More specifically, the ability of PGI₂-hMSCs to induce endogenous H19 lncRNA upregulation in primary myoblasts was investigated.

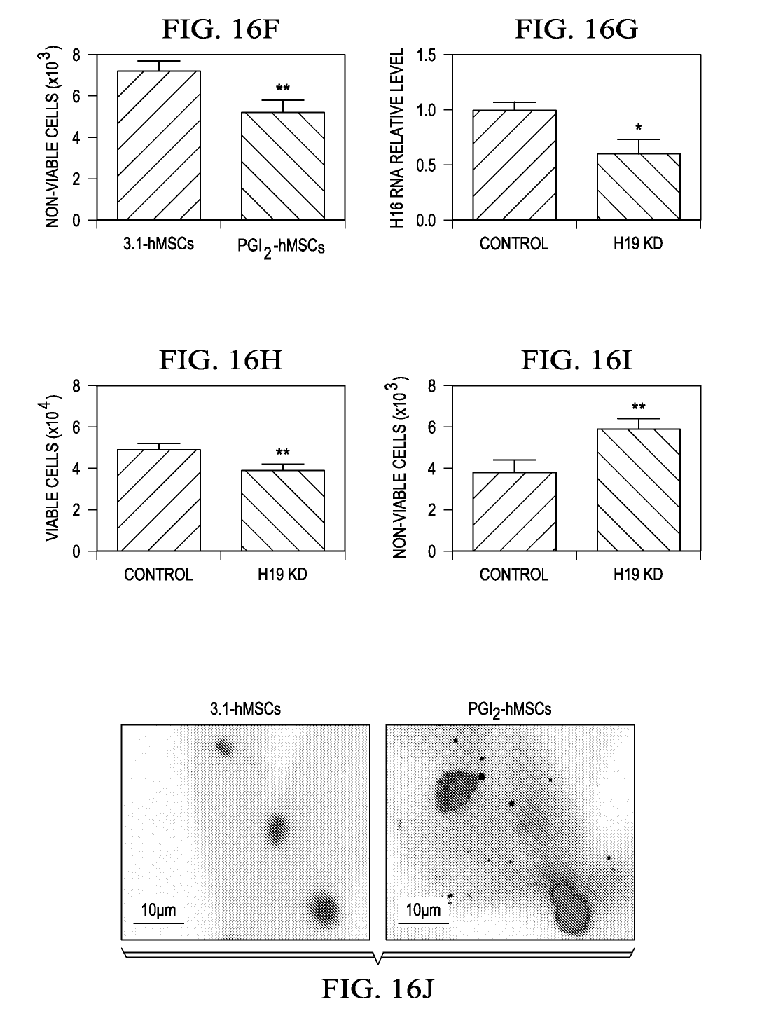

The effect of the secretome of PGI₂-hMSC (e.g., protective effect) on host progenitor cells was investigated. Thus, the in vitro coculturing assays were repeated as described in Example 10 with primary myoblasts isolated from the same strain of NOD/SCID mice used in functional assessments. Supporting the above results, a significant 2-fold increase was found in H19 transcript levels in myoblasts cocultured with PGI₂-hMSCs as compared with those cocultured with 3.1-hMSCs for 24 hours (P<0.05; FIG. 16A). FIG. 16A indicates that H19 RNA levels increased significantly in primary myoblasts cocultured with PGI₂-hMSCs for 24 hour in a hypoxic incubator as compared with 3.1-hMSC coculture.

Similarly, coculturing did not affect the number of viable cells (FIG. 16B) but significantly reduced the number of nonviable cells after 24 hours of PGI₂-hMSC coculture (3.57±0.27×10³ vs 5.00±0.35×10³ cells; myoblasts cocultured with PGI₂-hMSC vs 3.1-hMSC, respectively, P<0.02; FIG. 16C). PGI₂-hMSC coculture did not increase myoblast growth (FIG. 16B) but significantly reduced nonviable cells (FIG. 16C) compared with 3.1-hMSC coculture at 24 hours.

At 48 hours, the level of H19 RNA was comparable (FIG. 16D), but there was an 18% increase in viable cells (7.67±0.10×10⁴ vs 6.47±0.10×10⁴ cells; P<0.01; FIG. 16E) and a 27% decrease in nonviable cells (5.30±0.30×10³ vs 7.25±0.32×10³ cells; P<0.01; FIG. 16F) in PGI₂-hMSC-cocultured myoblasts as compared with those cocultured with 3.1-hMSCs. FIG. 16D indicates that H19 RNA levels were comparable in myoblasts cocultured for 48 hours with PGI₂-hMSCs or 3.1-hMSCs. Coculture with PGI₂-hMSCs for 48 hours induced a significant increase in viable myoblasts (FIG. 16E) and a significant decrease in nonviable cells (FIG. 16F) compared with coculture with 3.1-hMSCs.

The siRNA approach described in Example 10 was used to evaluate the survival or growth benefit of H19 in primary myoblasts. Downregulation of H19 RNA (40%, P<0.02) caused a 23% reduction of viable cells (4.89±0.16×10⁴ vs 3.97±0.09×10⁴ cells; negative control siRNA vs H19 siRNA-transfected myoblasts; P<0.01; FIG. 16H) and a 34% increase of nonviable cells (3.85±0.32×10³ vs 5.87±0.27×10³ cells; P<0.01; FIG. 16I). H19 silencing significantly reduced the number of viable cells (FIG. 16H) and increased cell death (FIG. 16I).

After confirming that PGI₂-hMSCs trigger H19 upregulation in host myoblasts in vitro during low oxygen tension, the effect of PGI₂-hMSCs was examined on endogenous H19 RNA expression in ischemic hindlimbs by using fluorescence in situ hybridization (RNA-FISH). A mouse H19 fluorescent oligonucleotide probe was used to detect single H19 RNA molecules. H19 sequences that contain miR-675-3p and miR-675-5p were excluded in the probe design to avoid non-specific binding. RNA-FISH studies showed an increase of H19 RNA levels in the cytoplasm and within the nucleus of endogenous cells surrounding PGI2-hMSC injection sites as compared with 3.1-hMSC injection sites at 3 days after cell administration (FIG. 16G). These results demonstrate endogenous H19 RNA upregulation induced by PGI₂-hMSCs. FIG. 16G indicated that H19 silencing by siRNA significantly reduced H19 levels in primary myoblasts.

FIG. 16J displays representative images of H19 RNA fluorescence in situ hybridization in gastrocnemius muscle sections at 3 days after 3.1-hMSC or PG₂-hMSC injections. A fluorescein-tagged H19 FISH probe that specifically targets endogenous H19 RNA was used, resulting in intense intracellular green fluorescent particles. A higher expression of host H19 RNA was found in PGI₂-hMSC-treated muscles than in 3.1-hMSC-treated muscles. All sections were counterstained with DAPI to localize nuclei. *P<0.05; **P<0.01.

### Additional Disclosure

The following are nonlimiting, specific embodiments in accordance with the present disclosure:

A first embodiment, which is an effective amount of a composition comprising a stem cell, a stem cell engraftment enhancer, and a carrier fluid, for use in the treatment of an individual having a disease or at risk of developing a disease, wherein the disease is a vascular-associated disease and/or a muscular disease.

A second embodiment, which is an effective amount of a composition comprising a stem cell, a stem cell engraftment enhancer, and a carrier fluid, for use in the treatment of an individual having a vascular-associated disease or at risk of developing a vascular-associated disease.

A third embodiment, which is an effective amount of a composition comprising a stem cell, a stem cell engraftment enhancer, and a carrier fluid, for use in the treatment of an individual having a muscular disease or at risk of developing a muscular disease.

A fourth embodiment, which is the composition of any of the first and the second embodiments wherein the vascular-associated disease comprises peripheral arterial disease, peripheral vascular disease, thrombosis, ischemia, critical limb ischemia, heart attack, acute myocardial infarction, congestive heart failure, pulmonary arterial hypertension, acute lung injury, stroke, inflammation in an organ or vessel of a vascular system, chronic kidney disease, leukemia, bone marrow transplant, metabolic diseases, diabetes, or combinations thereof.

A fifth embodiment, which is the composition of any of the first through the fourth embodiments wherein the stem cell comprises human mesenchymal stem cells (hMSCs), endothelial progenitor cells (EPCs), hematopoietic stem cells (HSCs), cardiac progenitor cells (CPCs), satellite cells, or combinations thereof.

A sixth embodiment, which is the composition of any of the first through the fifth embodiments wherein the stem cell overexpresses prostacyclin (PGI2).

A seventh embodiment, which is the composition of any of the first through the sixth embodiments wherein the stem cell engraftment enhancer comprises PGI2, a PGI2 precursor, a peroxisome proliferator-activated receptor β/δ isoform (PPARδ) agonist, a cAMP inducer, a phosphodiesterase inhibitor, an endothelin receptor antagonist, a nitrous oxide modulating agent, a prostacyclin receptor (IP) agonist, a non-prostanoid IP receptor agonist, or combinations thereof.

An eighth embodiment, which is the composition of the seventh embodiment wherein the PGI2 precursor comprises a triple catalytic enzyme, a PGI2-overexpressing stem cell (PGI2-SC), a DNA sequence encoding for the triple catalytic enzyme, a cDNA sequence encoding for the triple catalytic enzyme, a host cell containing an expressible DNA sequence encoding for the triple catalytic enzyme, a vector comprising a DNA sequence encoding for the triple catalytic enzyme, a plasmid comprising a DNA sequence encoding for the triple catalytic enzyme, a fusion gene encoding for the triple catalytic enzyme, a synthetic PGI2 analogue, or combinations thereof.

A ninth embodiment, which is the composition of the eighth embodiment wherein the synthetic PGI2 analogue is selected from the group consisting of Iloprost, Carbaprostacyclin, Treprostinil, Cicaprost, Beraprost, and Epoprostenol.

A tenth embodiment, which is the composition of the eighth embodiment wherein the triple catalytic enzyme is characterized by a formula COX-linker-ES, wherein COX comprises a cyclooxygenase (COX) amino acid sequence; ES comprises an eicosanoid-synthesizing (ES) enzyme amino acid sequence; and the linker comprises from about 10 to about 22 amino acid residues of a transmembrane sequence; wherein the linker is disposed between the COX and the ES, and wherein the linker directly connects the COX to the ES.

An eleventh embodiment, which is the composition of the any of the eighth through tenth embodiments wherein the triple catalytic enzyme is characterized by a formula COX-1-10aa-PGIS; wherein COX-1 is cyclooxygenase isoform-1; the linker comprises a 10 amino acid (10aa) transmembrane sequence; and PGIS is prostacyclin synthase.

A twelfth embodiment, which is the composition of any of the first through the eleventh embodiments further comprising a PGI2-overexpressing human mesenchymal stem cell (PGI2-hMSC).

A thirteenth embodiment, which is the composition of any of the first through the twelfth embodiments administered via an intramuscular injection.

A fourteenth embodiment, which is the composition of any of the first through the thirteenth embodiments having PGI2-SCs and a carrier fluid wherein the composition is administered via a single treatment stream.

A fifteenth embodiment, which is the composition of any of the first through fourteenth embodiments comprising stem cells, a PPARδ agonist, and a carrier fluid wherein the composition is administered via a single treatment stream.

A sixteenth embodiment, which is the composition of any of the first through the thirteenth embodiments administered via multiple treatment streams comprising:
a stem cell treatment stream and a stem cell engraftment enhancer treatment stream,
wherein the stem cell treatment stream comprises stem cells and a carrier fluid; and
wherein the stem cell engraftment enhancer treatment stream comprises a stem cell engraftment enhancer and a carrier fluid.

A seventeenth embodiment, which is the composition of the sixteenth embodiment wherein the stem cell treatment stream comprises hMSCs and wherein the stem cell engraftment enhancer treatment stream comprises PGI2, a PGI2 precursor, or both.

An eighteenth embodiment, which is the composition of any of the first through the seventeenth embodiments wherein a stem cell engraftment in an individual treated with the composition is enhanced by greater than about 200%, when compared to stem cell engraftment in an individual treated with a composition lacking the stem cell engraftment enhancer.

A nineteenth embodiment, which is the composition of any of the first through the eighteenth embodiments wherein the composition up-regulates a long non-coding RNA H19 in a host environment.

A twentieth embodiment, which is the composition of the nineteenth embodiment wherein up-regulating the long non-coding RNA H19 in the host environment promotes host cell growth.

A twenty-first embodiment, which is a composition comprising PGI2-overexpressing human mesenchymal stem cells (PGI2-hMSCs), and a carrier fluid; wherein an effective amount of the composition is administered via a single treatment stream as an intramuscular injection to an individual having a disease or at risk of developing a disease, wherein the disease is a vascular-associated disease and/or a muscular disease, and wherein stem cell engraftment is enhanced in said individual by greater than about 200%, when compared to stem cell engraftment in an individual treated with a composition lacking the stem cell engraftment enhancer.

A twenty-second embodiment, which is a composition comprising PGI2-overexpressing human mesenchymal stem cells (PGI2-hMSCs), and a carrier fluid; wherein an effective amount of the composition is administered via a single treatment stream as an intramuscular injection to an individual having a vascular-associated disease or at risk of developing a vascular-associated disease, and wherein stem cell engraftment is enhanced in said individual by greater than about 200%, when compared to stem cell engraftment in an individual treated with a composition lacking the stem cell engraftment enhancer.

A twenty-third embodiment, which is a composition comprising PGI2-overexpressing human mesenchymal stem cells (PGI2-hMSCs), and a carrier fluid; wherein an effective amount of the composition is administered via a single treatment stream as an intramuscular injection to an individual having a muscular disease or at risk of developing a muscular disease, and wherein stem cell engraftment is enhanced in said individual by greater than about 200%, when compared to stem cell engraftment in an individual treated with a composition lacking the stem cell engraftment enhancer.

A twenty-fourth embodiment, which is a composition comprising human mesenchymal stem cells (hMSCs), Iloprost, and a carrier fluid; wherein the composition is administered to an individual having a disease or at risk of developing a disease, wherein the disease is a vascular-associated disease and/or a muscular disease, and wherein the composition is administered via multiple treatment streams comprising: a stem cell treatment stream, and a stem cell engraftment enhancer treatment stream; wherein the stem cell treatment stream comprises hMSCs and is administered via an intramuscular injection; and wherein the stem cell engraftment enhancer treatment stream comprises Iloprost and is administered via inhalation.

A twenty-fifth embodiment, which is a composition comprising human mesenchymal stem cells (hMSCs), Iloprost, and a carrier fluid; wherein the composition is administered to an individual having a vascular-associated disease or at risk of developing a vascular-associated disease via multiple treatment streams comprising: a stem cell treatment stream, and a stem cell engraftment enhancer treatment stream; wherein the stem cell treatment stream comprises hMSCs and is administered via an intramuscular injection; and wherein the stem cell engraftment enhancer treatment stream comprises Iloprost and is administered via inhalation.

A twenty-sixth embodiment, which is a composition comprising human mesenchymal stem cells (hMSCs), Iloprost, and a carrier fluid; wherein the composition is administered to an individual having a muscular disease or at risk of developing a muscular disease via multiple treatment streams comprising: a stem cell treatment stream, and a stem cell engraftment enhancer treatment stream; wherein the stem cell treatment stream comprises hMSCs and is administered via an intramuscular injection; and wherein the stem cell engraftment enhancer treatment stream comprises Iloprost and is administered via inhalation.

A twenty-seventh embodiment, which is a composition for stem cell engraftment, wherein the composition for stem cell engraftment comprises a stem cell, wherein the stem cell comprises: human mesenchymal stem cells (hMSCs), endothelial progenitor cells (EPCs), hematopoietic stem cells (HSCs), cardiac progenitor cells (CPCs), satellite cells, or combinations thereof, a stem cell engraftment enhancer, wherein the stem cell engraftment enhancer comprises: prostacyclin (PGI2), a PGI2 precursor, a peroxisome proliferator-activated receptor β/δ isoform (PPARδ) agonist, a cAMP inducer, a phosphodiesterase inhibitor, an endothelin receptor antagonist, a nitrous oxide modulating agent, a prostacyclin receptor (IP) agonist, a non-prostanoid IP receptor agonist, or combinations thereof, and a carrier fluid.

A twenty-eighth embodiment, which is the composition of the twenty-seventh embodiment wherein the stem cells overexpress PGI2.

While embodiments of the invention have been shown and described, modifications thereof can be made by one skilled in the art without departing from the spirit and teachings of the invention. The embodiments described herein are exemplary only, and are not intended to be limiting. Many variations and modifications of the invention disclosed herein are possible and are within the scope of the invention. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). For example, whenever a numerical range with a lower limit, Rₗ, and an upper limit, Rᵤ, is disclosed, any number falling within the range is specifically disclosed. In particular, the following numbers within the range are specifically disclosed: R=Rₗ+k*(Rᵤ−Rₗ), wherein k is a variable ranging from 1 percent to 100 percent with a 1 percent increment, i.e., k is 1 percent, 2 percent, 3 percent, 4 percent, 5 percent, . . . 50 percent, 51 percent, 52 percent, . . . , 95 percent, 96 percent, 97 percent, 98 percent, 99 percent, or 100 percent. Moreover, any numerical range defined by two R numbers as defined in the above is also specifically disclosed. Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, etc.

Accordingly, the scope of protection is not limited by the description set out above but is only limited by the claims which follow, that scope including all equivalents of the subject matter of the claims. Each and every claim is incorporated into the specification as an embodiment of the present invention. Thus, the claims are a further description and are an addition to the embodiments of the present invention. The discussion of a reference in the Detailed Description of the Embodiments is not an admission that it is prior art to the present invention, especially any reference that may have a publication date after the priority date of this application. The disclosures of all patents, patent applications, and publications cited herein are hereby incorporated by reference, to the extent that they provide exemplary, procedural or other details supplementary to those set forth herein.

1. An effective amount of a composition comprising:
a stem cell, a stem cell engraftment enhancer, and a carrier fluid, for use in the treatment of an individual having a disease or at risk of developing a disease, wherein the disease is a vascular-associated disease and/or a muscular disease.

2. The composition of claim 1 wherein the vascular-associated disease comprises peripheral arterial disease, peripheral vascular disease, thrombosis, ischemia, critical limb ischemia, heart attack, acute myocardial infarction, congestive heart failure, pulmonary arterial hypertension, acute lung injury, stroke, inflammation in an organ or vessel of a vascular system, chronic kidney disease, leukemia, bone marrow transplant, metabolic diseases, diabetes, or combinations thereof.

3. The composition of claim 1 wherein the stem cell comprises human mesenchymal stem cells (hMSCs), endothelial progenitor cells (EPCs), hematopoietic stem cells (HSCs), cardiac progenitor cells (CPCs), satellite cells, or combinations thereof.

4. The composition of claim 1 wherein the stem cell overexpresses prostacyclin (PGI2).

5. The composition claim 1 wherein the stem cell engraftment enhancer comprises PGI2, a PGI2 precursor, a peroxisome proliferator-activated receptor β/δ isoform (PPARδ) agonist, a cAMP inducer, a phosphodiesterase inhibitor, an endothelin receptor antagonist, a nitrous oxide modulating agent, a prostacyclin receptor (IP) agonist, a non-prostanoid IP receptor agonist, or combinations thereof.

6. The composition of claim 5 wherein the PGI2 precursor comprises a triple catalytic enzyme, a PGI2-overexpressing stem cell (PGI2-SC), a DNA sequence encoding for the triple catalytic enzyme, a cDNA sequence encoding for the triple catalytic enzyme, a host cell containing an expressible DNA sequence encoding for the triple catalytic enzyme, a vector comprising a DNA sequence encoding for the triple catalytic enzyme, a plasmid comprising a DNA sequence encoding for the triple catalytic enzyme, a fusion gene encoding for the triple catalytic enzyme, a synthetic PGI2 analogue, or combinations thereof.

7. The composition of claim 6 wherein the synthetic PGI2 analogue is selected from the group consisting of Iloprost, Carbaprostacyclin, Treprostinil, Cicaprost, Beraprost, and Epoprostenol.

8. The composition of claim 6 wherein the triple catalytic enzyme is characterized by a formula COX-linker-ES, wherein COX comprises a cyclooxygenase (COX) amino acid sequence; ES comprises an eicosanoid-synthesizing (ES) enzyme amino acid sequence; and the linker comprises from about 10 to about 22 amino acid residues of a transmembrane sequence; wherein the linker is disposed between the COX and the ES, and wherein the linker directly connects the COX to the ES.

9. The composition of claim 8 wherein the triple catalytic enzyme is characterized by a formula COX-1-10aa-PGIS; wherein COX-1 is cyclooxygenase isoform-1; the linker comprises a 10 amino acid (10aa) transmembrane sequence; and PGIS is prostacyclin synthase.

10. The composition claim 1 further comprising a PGI2-overexpressing human mesenchymal stem cell (PGI2-hMSC).

11. The composition of claim 1 administered via an intramuscular injection.

12. The composition of claim 1 having PGI2-SCs and a carrier fluid wherein the composition is administered via a single treatment stream.

13. The composition of claim 1 comprising stem cells, a PPAR agonist, and a carrier fluid wherein the composition is administered via a single treatment stream.

14. The composition of claim 1 administered via multiple treatment streams comprising:
a stem cell treatment stream and a stem cell engraftment enhancer treatment stream,
wherein the stem cell treatment stream comprises stem cells and a carrier fluid; and
wherein the stem cell engraftment enhancer treatment stream comprises a stem cell engraftment enhancer and a carrier fluid.

15. The composition of claim 14 wherein the stem cell treatment stream comprises hMSCs and wherein the stem cell engraftment enhancer treatment stream comprises PGI2, a PGI2 precursor, or both.

16. The composition of claim 1 wherein a stem cell engraftment in an individual treated with the composition is enhanced by greater than about 200%, when compared to stem cell engraftment in an individual treated with a composition lacking the stem cell engraftment enhancer.

17. The composition of claim 1 wherein the composition up-regulates a long non-coding RNA H19 in a host environment.

18. The composition of claim 17 wherein up-regulating the long non-coding RNA H19 in the host environment promotes host cell growth.

19. A composition comprising PGI2-overexpressing human mesenchymal stem cells (PGI2-hMSCs), and a carrier fluid; wherein an effective amount of the composition is administered via a single treatment stream as an intramuscular injection to an individual having a disease or at risk of developing a disease, wherein the disease is a vascular-associated disease and/or a muscular disease, and wherein stem cell engraftment is enhanced in said individual by greater than about 200%, when compared to stem cell engraftment in an individual treated with a composition lacking the stem cell engraftment enhancer.

20. A composition comprising:
human mesenchymal stem cells (hMSCs),
Iloprost, and
a carrier fluid;
wherein the composition is administered to an individual having a disease or at risk of developing a disease, wherein the disease is a vascular-associated disease and/or a muscular disease, and wherein the composition is administered via multiple treatment streams comprising:
a stem cell treatment stream, and a stem cell engraftment enhancer treatment stream;
wherein the stem cell treatment stream comprises hMSCs and is administered via an intramuscular injection; and
wherein the stem cell engraftment enhancer treatment stream comprises Iloprost and is administered via inhalation.

21. A composition for stem cell engraftment, wherein the composition for stem cell engraftment comprises:
a stem cell, wherein the stem cell comprises:
human mesenchymal stem cells (hMSCs), endothelial progenitor cells (EPCs), hematopoietic stem cells (HSCs), cardiac progenitor cells (CPCs), satellite cells, or combinations thereof;
a stem cell engraftment enhancer, wherein the stem cell engraftment enhancer comprises:
prostacyclin (PGI2), a PGI2 precursor, a peroxisome proliferator-activated receptor β/δ isoform (PPARδ) agonist, a cAMP inducer, a phosphodiesterase inhibitor, an endothelin receptor antagonist, a nitrous oxide modulating agent, a prostacyclin receptor (IP) agonist, a non-prostanoid IP receptor agonist, or combinations thereof; and
a carrier fluid.

22. The composition of claim 21 wherein the stem cells overexpress PGI2.


SEQUENCE LISTING (2 sequences)

SEQ ID NO: 1 - protein, 10 aa, Artificial Sequence
  synthetic sequence
HAIMGVAFTW

SEQ ID NO: 2 - protein, 22 aa, Artificial Sequence
  synthetic sequence
HAIMGVAFTWVMALACAAPPLV